SEQUENCE LISTING (45 sequences)

SEQ ID NO: 1 - DNA, 21 bp, Artificial
  synthetic construct; 5′ PTEN primer
agcgtgcagataatgacaagg

SEQ ID NO: 2 - DNA, 21 bp, Artificial
  synthetic construct; 3′ PTEN primer
tggatcagagtcagtggtgtc

SEQ ID NO: 3 - DNA, 18 bp, Artificial
  synthetic construct; 5′ GAPDH primer
gaaggtgaaggtcggagt

SEQ ID NO: 4 - DNA, 20 bp, Artificial
  synthetic construct; 3′ GAPDH primer
gaagatggtgatgggatttc

SEQ ID NO: 5 - DNA, 23 bp, Artificial
  synthetic construct; 5′ HES1 primer
ctggaaatgacagtgaagcacct

SEQ ID NO: 6 - DNA, 22 bp, Artificial
  synthetic construct; 3′ HES1 primer
attgatctgggtcatgcagttg

SEQ ID NO: 7 - DNA, 23 bp, Artificial
  synthetic construct; 5′ DELTEX1 primer
aagaagttcaccgcaagaggatt

SEQ ID NO: 8 - DNA, 23 bp, Artificial
  Synthetic Construct; 3′ DELTEX1 primer
ctaggtagctagcgtccgggtag

SEQ ID NO: 9 - DNA, 20 bp, Artificial
  Synthetic Construct; 5′ Beta-actin primer
atggtgggaatgggtcagaa

SEQ ID NO: 10 - DNA, 20 bp, Artificial
  Synthetic Construct; 3′ Beta-actin primer
tctccatgtcgtcccagttg

SEQ ID NO: 11 - DNA, 18 bp, Artificial
  Synthetic Construct; 5′ Hes1 primer
tcctgacggccaatttgc

SEQ ID NO: 12 - DNA, 21 bp, Artificial
  Synthetic Construct; 3′ Hes1 primer
ggaaggtgacactgcgttagg

SEQ ID NO: 13 - DNA, 24 bp, Artificial
  Synthetic Construct; 5′ PTEN primer
ggggaagtaaggaccagagacaaa

SEQ ID NO: 14 - DNA, 22 bp, Artificial
  Synthetic Construct; 3′ PTEN primer
ccacgggtctgtaatccaggtg

SEQ ID NO: 15 - DNA, 22 bp, Artificial
  Synthetic Construct; 5′ PTEN primer A
aggtctcagtcctttggcttgc

SEQ ID NO: 16 - DNA, 22 bp, Artificial
  Synthetic Construct; 3′ PTEN primer A
tggttacacaagcacccacatc

SEQ ID NO: 17 - DNA, 22 bp, Artificial
  Synthetic Construct; 5′ PTEN primer B
gtgatgtggcgggactctttat

SEQ ID NO: 18 - DNA, 21 bp, Artificial
  Synthetic Construct; 3′ PTEN primer B
ctctcatctccctcgcctgag

SEQ ID NO: 19 - DNA, 22 bp, Artificial
  Synthetic Construct; 5′ PTEN primer C
gtgatgtggcgggactctttat

SEQ ID NO: 20 - DNA, 21 bp, Artificial
  Synthetic Construct; 3′ PTEN primer C
ctctcatctccctcgcctgag

SEQ ID NO: 21 - DNA, 18 bp, Artificial
  Synthetic Construct; 5′ Beta-Actin genomic primer
agcgcggctacagcttca

SEQ ID NO: 22 - DNA, 25 bp, Artificial
  Synthetic Construct; 3′ Beta-Actin genomic primer
cgtagcacagcttctccttaatgtc

SEQ ID NO: 23 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 24 - protein, 403 aa, Homo sapiens [sequence omitted]

SEQ ID NO: 25 - DNA, 12 bp, Artificial
  Synthetic Construct
gggccgtggacg

SEQ ID NO: 26 - DNA, 36 bp, Artificial
  Synthetic Construct
cgcgccttccccacaacagctccttccacttcctgc

SEQ ID NO: 27 - DNA, 42 bp, Artificial
  Synthetic Construct
cccggttgggcagcctcaacatcccctacaagatcgaggccg

SEQ ID NO: 28 - DNA, 11 bp, Artificial
  Synthetic Construct
gcctcttgcat

SEQ ID NO: 29 - DNA, 24 bp, Artificial
  Synthetic Construct
ccgtggagctgatgccgccggagc

SEQ ID NO: 30 - protein, 34 aa, Artificial
  Synthetic Construct
CCSHWAPAAWRCTLFCPRRAPPCPRRCHPRWSHP

SEQ ID NO: 31 - protein, 38 aa, Artificial
  Synthetic Construct
EGRGRCSHWAPAAWRCTLFCPRRAPPCPRRCHPRWSHP

SEQ ID NO: 32 - protein, 12 aa, Artificial
  Synthetic Construct
PRLPHNSSFHFL

SEQ ID NO: 33 - protein, 14 aa, Artificial
  Synthetic Construct
RLGSLNIPYKIEAV

SEQ ID NO: 34 - protein, 99 aa, Artificial
  Synthetic Construct
ASCILWTTPPATSYRCLSTPSSPRPLSPLTSGPARPRIPTSPTGPRASPALPPACSPRSPAFRRPSSKRRAPRDPGFLSQAFGRLCALCGCQGRPEEPF

SEQ ID NO: 35 - protein, 8 aa, Artificial
  Synthetic Construct
PVELMPPE

SEQ ID NO: 36 - RNA, 10 nt, Artificial
  Synthetic Construct
cccccggccc

SEQ ID NO: 37 - DNA, 9 bp, Artificial
  Synthetic Construct
agagcccct

SEQ ID NO: 38 - DNA, 11 bp, Artificial
  Synthetic Construct
cattctgtacc

SEQ ID NO: 39 - DNA, 12 bp, Artificial
  Synthetic Construct
gggtatatactt

SEQ ID NO: 40 - DNA, 10 bp, Artificial
  Synthetic Construct
ctttgagttc

SEQ ID NO: 41 - DNA, 10 bp, Artificial
  Synthetic Construct
cgacgggaag

SEQ ID NO: 42 - DNA, 13 bp, Artificial
  Synthetic Construct
aggggctgggcaa

SEQ ID NO: 43 - DNA, 20 bp, Artificial
  Synthetic Construct
gggcgagggagtatgaggcc

SEQ ID NO: 44 - protein, 33 aa, Artificial
  Synthetic Construct
CSHWAPAAWRCTLFCPRRAPPCPRRCHPRWSHP

SEQ ID NO: 45 - DNA, 18 bp, Artificial
  Synthetic Construct
cagatgatcttcccctac

(19) United States
(12) Patent Application Publication
(10) Pub. No.: US 2011/0118192 A1
(43) Pub. Date: May 19, 2011

(54) METHODS AND COMPOSITIONS FOR TREATING T-CELL LEUKEMIA

(75) Inventors: Adolfo A. Ferrando, New York, NY (US); Teresa Palomero, New York, NY (US); Maria Luisa Sulis, New York, NY (US)

(73) Assignee: TRUSTEES OF COLUMBIA UNIVERSITY IN THE CITY OF NEW YORK, New York, NY (US)

(21) Appl. No.: 12/449,291

(22) PCT Filed: Feb. 1, 2008
(86) PCT No.: PCT/US08/01434

§ 371 Date: Jan. 31, 2011

Related U.S. Application Data
(60) Provisional application No. 60/899,179, filed on Feb. 1, 2007.

(51) Int. Cl. A61K 38/05; C12Q 1/68; G01N 33/53; C40B 30/04; G01N 33/68; C12Q 1/02; A61K 31/5513; A61P 35/02; A61P 35/00; A61K 31/661

(52) U.S. Cl. 514/19.4; 435/6; 436/94; 506/9; 436/86; 435/7.92; 435/29; 514/221; 514/19.3; 514/19.6; 514/129

(57) ABSTRACT

The present invention relates to compositions and methods that may be used to diagnose and treat cancer, particularly T-cell leukemia. According to one preferred embodiment of the present invention, methods are provided for determining whether reducing or blocking NOTCH-1 activation will be effective to treat, prevent, or ameliorate the effects of a cancer in a patient, including T-cell leukemia, myeloleukemia, neuroblastoma, breast cancer, and ovarian cancer. The methods generally include determining if the patient harbors one or more mutations in a PTEN coding region. In particular, the methods may be used to determine whether reducing or blocking NOTCH-1 activation, with one or more γ-secretase inhibitors, will be effective to treat, prevent, or ameliorate the effects of a cancer in a patient.

### CROSS-REFERENCE TO RELATED APPLICATIONS

This application claims benefit to U.S. provisional patent application Ser. No. 60/899,179, filed Feb. 1, 2007, which is incorporated by reference in its entirety as if recited in full herein.

### FEDERALLY SPONSORED RESEARCH OR DEVELOPMENT

The invention was made in part with government support under grant number CA120196 awarded by the National Institutes of Health. The government may have certain rights in the invention.

### FIELD OF THE INVENTION

The present invention relates to compositions and methods that may be used to diagnose and treat cancer, particularly T-cell leukemia.

### BACKGROUND OF THE INVENTION

NOTCH receptors directly transduce extracellular signals at the cell surface into changes in gene expression that regulate differentiation, self-renewal, proliferation and apoptosis. Constitutively active forms of the NOTCH-1 receptor contribute to over 50% of human T-cell lymphoblastic leukemias and lymphomas (“T-ALL”), and have also been implicated in the pathogenesis of solid tumors, such as breast carcinomas, gliomas and neuroblastoma. NOTCH-1 signaling, whether initiated by receptor-ligand interactions or triggered by mutations in the NOTCH-1 gene, requires two consecutive proteolytic cleavages in the receptor, the first by an ADAM metalloprotease and the second by a γ-secretase complex. The final cleavage releases intracellular NOTCH-1 from the membrane, which then translocates to the nucleus and interacts with the CSL DNA-binding protein (a transcription factor) to activate the expression of target genes. The high prevalence of activating mutations in NOTCH-1 in T-ALL and the availability of small molecule inhibitors of γ-secretase (GSIs) capable of blocking NOTCH-1 activation, have prompted clinical trials to test the effectiveness of these agents against T-ALL.

However, the efficacy of this strategy has been questioned as GSIs seem to be active in only a small fraction of T-ALL cell lines with constitutive NOTCH-1 activity. In light of the foregoing, there is a need for methods and compositions that enable clinicians to identify T-ALL cell lines, and patients harboring such cell lines, which will be responsive to GSI activity.

### SUMMARY OF THE INVENTION

According to certain preferred embodiments of the present invention, methods are provided for determining whether reducing or blocking NOTCH-1 activation will be effective to treat, prevent, or ameliorate the effects of a cancer in a patient. The methods generally comprise determining if the patient harbors one or more mutations in a PTEN coding region.

According to another preferred embodiment of the present invention, methods are provided for determining whether an AKT inhibitor will be effective to treat, prevent, or ameliorate the effects of a cancer in a patient comprising determining if the patient harbors one or more mutations in a PTEN coding region.

According to certain related embodiments of the invention, methods are provided for treating, preventing, or ameliorating the effects of a cancer in a patient comprising determining if the patient harbors one or more mutations in a PTEN coding region and (a) providing the patient with an AKT inhibitor if the patient harbors such mutations or (b) reducing or blocking NOTCH-1 activation in the patient if the patient does not harbor such mutations.

According to further embodiments of the invention, methods for identifying whether a patient is resistant to a γ-secretase inhibitor are provided. Such methods generally comprise determining whether the patient has a mutation in a PTEN gene.

According to still further embodiments of the invention, methods are provided for identifying a patient population for inclusion in a clinical trial of a drug candidate for treating cancer. Such methods generally comprise carrying out a screen for PTEN mutations on a sample of DNA from each prospective patient, wherein the presence of a PTEN mutation in a patient's DNA sample is indicative of that patient being resistant to γ-secretase inhibitors and sensitive to AKT inhibitors. Such methods further comprise determining whether to include each patient in the clinical trial based on the patient's PTEN mutation status determined by the screen and the mode of action of the drug candidate.

### BRIEF DESCRIPTION OF THE FIGURES

FIG. 1. PTEN loss and AKT activation in GSI-resistant T-ALLs. (a) Nearest Neighbor analysis of genes associated with GSI sensitivity and resistance in T-ALL cell lines. Relative gene expression levels are color coded with lighter colors (higher levels of expression) and darker colors (lower levels of gene expression). (b) Western blot analysis of PTEN and p-AKT (Ser473) in T-ALL cell lines. AKT and α-tubulin are shown as loading controls. (c) Representative images of PTEN immunostaining in T-cell lymphoblastic tumors showing diffuse negative staining with scattered positive cells (arrowheads) in a PTEN negative sample (upper panel), cytoplasmic PTEN expression in a PTEN positive sample (lower panel). (d) Schematic representation of PTEN mutations identified in TALL samples.

FIG. 2. PTEN loss and AKT activation induce GSI resistance in T-ALL. (a and b) Decreased cell size (FSC-H) and decreased cell growth induced by GSI treatment (CompE 100 nM for 4 days) are rescued by retroviral expression of a constitutive active AKT (Myr-AKT) in CUTLL1 cells. (c and d) shRNA knock-down of PTEN restores cell size defects and reduced cell growth of DND41 cells treated with GSI (CompE 100 nM for 4 days) compared to that of vehicle (DMSO) treated controls. No protective effect was observed by expression of a control shRNA targeting the luciferase gene (shRNA LUC). Mean FSC-H values for GSI and vehicle only treatment controls are indicated. Bar graphs represent means +/− standard deviation of triplicate samples. P values were derived from Student's t-test.

FIG. 3. NOTCH1 regulates PTEN expression, AKT signaling and glucose metabolism. (a) Real-time PCR analysis of PTEN transcript levels upon NOTCH1 inhibition by GSI in CUTLL1 and HPB-ALL relative to (DMSO) controls. GAPDH levels were used as reference control. (b) Western blot analysis of PTEN and p-AKT (Ser473) in GSI sensitive T-ALL cell lines treated with CompE. AKT and α-Tubulin are shown as loading controls. (c) Real-time PCR analysis of Hes1 and PTEN expression in mouse DN3 thymocytes cocultured with stromal cells (OP9) or stromal cells expressing the NOTCH1 ligand Delta-like-1 (OP9-DL1). Data are means +/− standard deviation of duplicate (day 1) and triplicate (day 2) experiments. (d) Glucose uptake analysis in HPB-ALL and P12ICHIKAWA T-ALL cell lines in basal conditions (vehicle treatment only). (e) Glucose oxidation analysis in HPB-ALL and P12-ICHIKAWA T-ALL cell lines in basal conditions (vehicle treatment only). (f) Effects of GSI treatment in glucose uptake in HPB-ALL and P12-ICHIKAWA T-ALL cells. (g) Effects of GSI treatment in glucose oxidation in HPB-ALL and P12-ICHIKAWA T-ALL cells. Data shown in (d) and (f) are means +/− standard deviation of triplicates. Data shown in (e) and (g) are means +/− standard deviation of duplicates. P values in (a) and (c)-(g) were derived from Student's t-test.

FIG. 4. HES1 and MYC regulate PTEN expression downstream of NOTCH1. (a) Quantitative ChIP analysis of HES1 binding to PTEN promoter sequences. (b) Quantitative ChIP analysis of c-MYC binding to PTEN promoter sequences. Data are means +/− standard deviation of triplicates. TIS: transcription initiation site. (c) Effects of HES1 and MYC expression in PTEN promoter activity. Luciferase reporter assays were performed in 293T cells with a 2,666 base pair PTEN promoter construct (pGL3 PTEN HindIII-NotI). Data are means +/− standard deviation of triplicates. (d) Lentiviral shRNA knock-down of HES1 in CUTLL1 cells induces transcriptional upregulation of PTEN. Expression of a control shRNA targeting the luciferase gene (shRNA LUC) was used as control.

FIG. 5. Conservation of the NOTCH-PTEN-AKT regulatory axis in growth control and tumorigenesis in Drosophila. (a) Female wild type eye size. (b) Generalized expression of Delta by the eye-specific driver eyeless (ey)-Gal4 results in mild eye overgrowth (genotype ey-Gal4>UAS-DI). (c and d) Co-overexpression of Delta and Akt1 in the developing eye results in massive eye overgrowth (100%, n>200 flies) (c) and secondary eye growths (metastases) in distant tissues within the thorax (7.69% of flies, n=118) (d, white arrow). (e) Inhibition of NOTCH receptor proteolysis by non-lethal doses (1 mM) of the GSI DAPT inhibits Delta-induced overgrowth and results in flies with eyes and wings smaller than wild type (FIG. 16). (f) Gain of PTEN (using the transgene UAS-PTEN) results in strong suppression of Delta-mediated eye overgrowth (genotype of the fly shown is ey-Gal4>UAS-DI/UAS-PTEN). (g) Overexpression of fringe (UAS-fang), a NOTCH pathway modulator, results in NOTCH inhibition in the eye and hence a small eye defect. (h) Gain of expression of Akt1 gene using the GS1D233C P-element fully rescued eye growth defect caused by reducing NOTCH pathway activation (genotype ey-Gal4>UAS-fang/+; GS1D233 (Akt1)/+ (see FIGS. 13 and 14).

FIG. 6. Transcriptional networks downstream of NOTCH1 in T-ALL and effects of pharmacologic inhibition of AKT in T-ALL cells. (a) Schematic representation of the transcriptional regulatory networks controlling cell growth downstream of NOTCH1 in PTEN-positive/GSI-sensitive and PTEN-null/GSI-resistant T-ALL cells. The dashed arrow indicates a weak positive effect of MYC on PTEN expression compared with the strong negative transcriptional effects of HES1 in the promoter of this gene. (b) Relative cell growth of GSI-sensitive/PTEN-positive and GSI-resistant/PTEN-null T-ALL cell lines treated with the SH6 AKT inhibitor at 10 μM concentration for 72 hours. Data are means +/− standard deviation of triplicates.

FIG. 7. CompE treatment effectively blocks NOTCH1 processing in GSI-sensitive and resistant T-ALL cells. Western blot analysis of activated NOTCH1 (NOTCH1IC) levels in GSI-sensitive and resistant cell lines after 24 hours treatment with Comp E (100 nM). α-Tubulin levels are shown as loading control. Lower molecular weight bands correspond to activated NOTCH1 protein in cell lines harboring mutations that result in C-terminal truncations of the PEST domain (NOTCH 1IC-ΔPEST).

FIG. 8. CompE treatment effectively down regulates the expression of DELTEX1 in GSI-sensitive and resistant T-ALL cells. (a and b) Quantitative RT-PCR analysis of DELTEX1 expression in GSI-sensitive and resistant cell lines after 24 hours of treatment with Comp E (100 nM). Relative expression levels were calculated by the ΔΔCt method using GAPDH as a reference control. Data are means +/− standard deviation.

FIG. 9. Lentiviral shRNA knock-down of NOTCH1 effectively blocks NOTCH1 signaling in T-ALL cells and inhibits cell growth. (a) Western blot analysis of activated NOTCH1 levels in CCRF-CEM cells infected with lentiviral particles (pLKO puro) driving expression of shRNAs targeting NOTCH1 (shRNA NOTCH1) or the luciferase gene (shRNA LUC) used as a control. Five days after puromycin selection, cells were analyzed for the presence of activated NOTCH1 protein by Western blot analysis using the NOTCH1 Val1744 antibody (Cell Signaling Technologies). (b) Quantitative RT-PCR analysis of expression of the NOTCH1 target gene DELTEX1 in CCRF-CEM cells expressing shRNAs targeting NOTCH1 (shRNA NOTCH1) or the luciferase gene (shRNA LUC) used as a control. Data are means +/− standard deviation of triplicate measurements. (c) NOTCH1 shRNA in CUTLL1 cells induced a reduction in cell diameters as determined by flow cytometry compared to shRNA LUC infected controls.

FIG. 10. Expression of constitutively active AKT and shRNA knock down of PTEN in T-ALL cells. (a) Western blot analysis of AKT Ser473 phosphorylation in CUTLL1 cells infected with retroviral particles driving expression of myristoylated AKT and EGFP (pMIG MYR-AKT) or EGFP alone (pMIG) used as a control. (b) Western blot analysis of PTEN in DND41 cells infected with lentiviral particles driving the expression of a shRNA against PTEN (pGK-GFP shRNA PTEN) or the luciferase gene (pGK-GFP shRNA LUC) used as a control. α-Tubulin levels are shown as the loading control.

FIG. 11. Inhibition of NOTCH1 signaling with GSI induces autophagy in CUTLL1 cells. (a, b) Transmission electron microscopy analysis of an early pass culture of CUTLL1 cells treated with vehicle control (DMSO) or GSI (500 mM CompE) for 6 days. White arrow heads indicate phagosomes. Black arrowheads indicate mitochondria. (c) Quantitation of double membrane structures excluding mitochondria in control (DMSO) and GSI (CompE)-treated cells. Horizontal lines indicate the median. P values were derived from the Wilcoxon rank sum test. (d) Western blot analysis of the phagosome-associated LC3 protein in CUTLL1 cells treated with DMSO or CompE for 48 hours. Inhibition of NOTCH1 signaling with GSI induced the LC3-II isoform characteristic of cells undergoing macroautophagy.

FIG. 12. ChIP-on-chip analysis of NOTCH1, HES1 and MYC binding to PTEN promoter sequences. Schematic representation of the PTEN proximal promoter sequence indicating the location of the oligonucleotide probes (grey boxes) in the Agilent Proximal Promoter Arrays and the binding ratios obtained in HPBALL cells after hybridization of duplicate chromatin immunoprecipitation samples performed with antibodies against MYC (N262, Santa Cruz Biotechnology), HES1 (H140, Santa Cruz Biotechnology) and NOTCH1 (Val1744 antibody, Cell Signaling Technologies). TIS: transcription initiation site.

FIG. 13. Analysis of the growth phenotype associated with Akt1 gain- and loss-of-function in a Delta gain-of-function background. (a) Map of the Akt1 region and the insertion of the GS1D233C P-element. Blue boxes (dark) represent 5′ and 3′ UTR regions and red (light) boxes represent coding exons in the Akt1 gene. (b) Adult mutant fly carrying a secondary eye-derived growth (metastasis, arrow) within the abdomen (genotype ey-Gal4>DI/+; UASdDp110/+). (c) Transversal section through the abdomen of the fly in (b). Arrows point to eye-derived cells (marked by the red (light) pigment of the eye) infiltrating surrounding tissues. (c) Example of eye imaginal disc in which Delta and Akt1 genes are overexpressed using the UAS-DI transgene and the GS1D233C (Akt1) line. Note that cooperation between Delta-NOTCH and Akt pathways leads to massive eye tumor growth (compare with eye overgrowth caused by single overexpression of Delta in (i)). (d) Example of eye imaginal disc in which Delta and Akt1 genes are overexpressed using the UAS-DI transgene and the 1D233C (Akt1) GS line. Note that cooperation between Delta-NOTCH and AKT pathways leads to massive eye tumor growth. (e)-(i). Homozygous mutant Akt1⁻/Akt1⁻ clones were induced by (e)-(h) hsp70-Flp or (i) ey-Flpmediated recombination. Clones are labeled by the lack of GFP (e)-(h) or lacZ (i). The associated Akt1^{+/+} sibling clones are distinguished by the stronger staining of GFP or lacZ. Note that effects of Akt1 in cell proliferation are epistatic to the gain of Delta. Mitotic cells are very rarely found within the mutant clones, and mitotic cells are often found at the border of the clone. Arrows in (g) point to GFP-positive cells labeled by pH3 mitotic marker. Arrowhead in (i) points to a slightly larger Akt1⁻ clone in the Delta overexpression background. (j) Quantification of total eye discs clonal areas of +/+; Akt1⁻/Akt1⁻ (white bar) or ey-Gal4>DI/+; Akt1⁻/Akt1⁻ (grey bar) compared to control siblings clones (+/+; +/+, white bar) or (ey-Gal4>DI; +/+, grey bar). Sizes shown represent the mean of total clone measurement in wt (n=14) and ey-Gal4>DI (n=21) eye mosaic discs. Error bars show standard error measurement. P values were calculated by the unpaired Student's t-test (*, P=0.0487; ***, P<0.0001).

FIG. 14. Invasive metastatic eye-derived tumor tissue in flies with a Pi3K92E plus Delta gain-of-function background. (a) Transversal sections through a control wild type thorax with focus on dorso-longitudinal (DLM) and dorso-ventral (DVM) muscles, gut and salivary glands (Sgl). (b)-(c) Section through the thorax (b) and schematic representation (c) in a fly that co-expressed DI and the PI3K-Dp110 (genotype ey-Gal4>DI/+; UAS-dDₚ110/+) showing invading eye-derived secondary growth. (d) Detail of the secondary eye growth showing invasive behavior (black arrowheads). Scale bars represent 100 μm.

FIG. 15. Analysis of Akt phosphorylation by Delta. (a) Confocal images of third instar larval eye imaginal discs staining with p-Akt (Ser505). Anterior is to the right. Arrow denotes the increased staining of pAkt anterior to the front of retinal differentiation (Elav staining in red (lighter color)). Staining is also augmented around the ommatidia. (b) Note the enhanced staining of p-Akt in the ventral region of ey-Gal4>UAS-DI/+; Akt(GS1D233C)/+ disc. (c),(d) Early ectopic expression of Delta in clones induces overgrowth and p-Akt1 (images in (c) and (d) show a single confocal section). (e) Mosaic disc harboring Akt1 clones show higher p-Akt expression (arrows) cell-autonomously. (f) The same image showing the p-Akt channel.

FIG. 16. Inhibition of NOTCH signaling with GSI can suppress overgrowth caused by overexpression of Delta in vivo. (a) Quantification of NOTCH-like eye growth phenotypes associated with treatment with the presenilin/γ-secretase inhibitor DAPT in flies that overexpressed the NOTCH ligand Delta, during the proliferation phase of eye development. Genotype is ey-Gal4 UAS-DI (hereafter, ey-Gal4>DI). (b) Quantification of other NOTCH-like wing phenotypes (loss of wing margin and small wing size) in the ey-Gal4>DI treated animals. For each of the DAPT concentrations shown, the number of eyes and wings quantified were: n=215 (0 mM of DAPT), n=202 (0.25 mM), n=455 (0.5 mM) and n=318 (1 mM). Representative data from two independent experiments are shown.

FIG. 17. Forced expression of PTEN in PTEN-null/GSI-resistant T-ALL cells induces impaired cell growth and decreased proliferation. (a) Cell size analysis by flow cytometry of P12 ICHIKAWA T-ALL cells infected with retroviruses expressing GFP (control) or a bicistronic transcript encoding PTEN and GFP. (b) Cell cycle distribution of P12 ICHIKAWA GFP- and PTEN IRES GFP-infected cells. Expression of PTEN was associated with decreased cell size (a) and G1 cell cycle arrest (b).

FIG. 18. Table of NOTCH1 mutations in T-ALL cell lines. “HD” refers to the heterodimerization domain.

FIG. 19. Table of PTEN mutational analysis in T-ALL cell lines.

FIG. 20. Table of immunohistochemistry analysis of PTEN in T-cell leukemia and lymphoma samples.

FIG. 21. Table of PTEN mutational analysis in primary T-ALL samples.

FIG. 22. Table of PTEN mutation analysis in paired diagnostic and relapsed T-ALL samples.

FIG. 23. Nucleic acid sequences used as PCR primers.

### DETAILED DESCRIPTION OF THE INVENTION

According to certain preferred embodiments of the present invention, methods are provided for determining whether reducing or blocking NOTCH-1 activation will be effective to treat, prevent, or ameliorate the effects of a cancer in a patient, including T-cell leukemia, myeloleukemia, neuroblastoma, breast cancer, and ovarian cancer. The methods generally comprise determining if the patient harbors one or more mutations in a PTEN coding region. In particular, the methods may be used to determine whether reducing or blocking NOTCH-1 activation, with one or more γ-secretase inhibitors, will be effective to treat, prevent, or ameliorate the effects of a cancer in a patient. Non-limiting examples of such γ-secretase inhibitors include [(2S)-2-{[(3,5-Difluorophenyl)acetyl]amino}-N-[(3S)1-methyl-2-oxo-5-phenyl-2,3-dihydro-1H-1,4-benzodiazepin-3-yl]propanamide], N4N-(3,5-difluorophenacetyl)-L-alanyl]-Sphenylglycine-t-butylester, and analogs, salts, and combinations thereof.

The methods of the present invention provide that mutations in a PTEN coding region may be detected using any method well-known to those of ordinary skill in the art. For example, PTEN mutations may be detected by (a) extracting DNA from a patient, (b) amplifying a portion of the DNA that comprises the PTEN coding region to produce an amplicon, and (c) sequencing the amplicon and determining whether the amplicon comprises one or more mutations in the PTEN coding region. A representative amplicon may be produced using a pair of PCR primers consisting of, e.g., SEQ ID NO:1 and SEQ ID NO:2. Alternatively, one or more mutations in a PTEN coding region may be detected by (a) extracting DNA from the patient and (b) determining whether portions of the DNA in which the PTEN coding region resides hybridizes under standard conditions to one or more polynucleotides that are complementary to mutated forms of the PTEN coding region. Such hybridization procedures may be carried out using southern blot techniques or microarray analysis. Still further, one or more mutations in a PTEN coding region may be detected by (a) extracting DNA from a patient and (b) determining whether portions of the DNA in which the PTEN coding region resides hybridizes under standard conditions to one or more polynucleotides that are complementary to normal forms of PTEN, which also may involve the use of a southern blot or microarray analysis.

“Standard conditions” for hybridization mean in this context the conditions which are generally used by a person skilled in the art to detect specific hybridization signals, or preferably so called stringent hybridization and non-stringent washing conditions or more preferably so called moderately stringent conditions or even more preferably so called stringent hybridization and stringent washing conditions a person skilled in the art is familiar with. A specific example thereof is DNA which can be identified by subjecting it to high stringency hybridization using the digoxigenin (referred to as DIG hereinafter) DNA Labeling and detection kit (Roche Diagnostics, Tokyo, Japan) following the protocol given by the manufacturer. The hybridization solution contains 50% formamide, 5×SSC (10×SSC is composed of 87.65 g of NaCl and 44.1 g of sodium citrate in 1 liter), 2% blocking reagent (Roche Diagnostics, Tokyo, Japan), 0.1% N-lauroylsarcosine, and 0.3% sodium dodecyl sulfate (referred as to SDS hereinafter). Hybridization can be done overnight at 42° C. and then washing twice in 2×SSC containing 0.1% SDS for 5 minutes at room temperature and twice in 0.1×SSC containing 0.1% SDS for 15 minutes at 50° C. to 68° C. Detection can be done as indicated by manufacturer.

In still further embodiments of the invention, one or more mutations in a PTEN coding region may be detected by (a) reverse transcribing RNA that has been isolated from a patient into cDNA and (b) sequencing the cDNA and determining whether the amplicon comprises one or more mutations in the PTEN coding region. Alternatively, the presence or absence of one or more mutations in a PTEN coding region may be determined using protein-based assays. For example, PTEN protein levels may be measured in a body fluid that is obtained from the patient. As discussed further below, many of the PTEN mutations introduce stop codons into the coding region thereof, thereby inhibiting the full expression of such region. Accordingly, if a protein-based assay does not detect normal levels of PTEN, it may be inferred that the patient harbors one or more mutations in the PTEN coding region. Such protein levels may be measured in a body fluid harvested from the patient using, e.g., immunoblots, ELISAs, RIAs, flow cytometry, and combinations thereof.

According to further preferred embodiments of the present invention, methods are provided for determining whether an AKT inhibitor will be effective to treat, prevent, or ameliorate the effects of a cancer in a patient comprising determining if the patient harbors one or more mutations in a PTEN coding region. Non-limiting examples of such AKT inhibitors include phosphatidylinositol analogs, such as the AKT inhibitor III (a.k.a. SH-6).

In certain related embodiments of the invention, methods are provided for treating, preventing, or ameliorating the effects of a cancer in a patient comprising determining if the patient harbors one or more mutations in a PTEN coding region and (a) providing the patient with an AKT inhibitor if the patient harbors such mutations or (b) reducing or blocking NOTCH-1 activation in the patient if the patient does not harbor such mutations. In such embodiments of the invention, NOTCH-1 activation may be reduced or blocked by providing the patient with one or more γ-secretase inhibitors, such as [(2S)-2-{[(3,5-Difluorophenyl)acetyl]amino)-N-[(3S)1-methyl-2-oxo-5-phenyl-2,3-dihydro-1H-1,4-benzodiazepin-3-yl]propanamide], N-[N-(3,5-difluorophenacetyl)-L-alanyl]-Sphenylglycine-t-butylester, or analogs, salts, or combinations thereof. If the patient harbors one or more mutations in the PTEN coding region, phosphatidylinositol analogs, e.g., SH-6, may be provided to the patient as an AKT inhibitor. The methods of such embodiments of the invention may be used for treating, preventing, or ameliorating the effects of T-cell leukemia, myeloleukemia, neuroblastoma, breast cancer, and/or ovarian cancer.

According to still further embodiments of the invention, methods for identifying whether a patient is resistant to a γ-secretase inhibitor are provided. Such methods generally comprise determining whether the patient has a mutation in a PTEN gene. The presence or absence of a mutation in the PTEN gene may be carried out using a high-throughput screening assay. Similar aspects of the invention include methods for identifying whether a patient is sensitive to an AKT inhibitor. These methods generally comprise carrying out a screen for PTEN mutations on a sample of DNA from a patient, wherein the presence of a PTEN mutation in the DNA sample is indicative of the patient being sensitive to an AKT inhibitor.

According to yet further embodiments of the invention, methods are provided for identifying a patient population for inclusion in a clinical trial of a drug candidate for treating cancer. Such methods generally comprise carrying out a screen for PTEN mutations on a sample of DNA from each prospective patient, wherein the presence of a PTEN mutation in a patient's DNA sample is indicative of that patient being resistant to γ-secretase inhibitors and sensitive to AKT inhibitors. Such methods further comprise determining whether to include each patient in the clinical trial based on the patient's PTEN mutation status determined by the screen and the mode of action of the drug candidate.

The following examples are provided to further illustrate the methods and compositions of the present invention. These examples are illustrative only and are not intended to limit the scope of the invention in any way.

### EXAMPLES

Inhibitors.

Compound E (CompE) [(2S)-2-{[(3,5-Difluorophenyl)acetyl]amino}-N-[(3S)1-methyl-2-oxo-5-phenyl-2,3-dihydro-1H-1,4-benzodiazepin-3-yl]propanamide] (Alexis Biochemicals) is a cell permeable, potent, selective, non-transition state and non-competitive inhibitor of γ-secretase. DAPT, N-[N-(3,5-difluorophenacetyl)-L-alanyl]-Sphenylglycine-t-butylester (Sigma-Aldrich) is a highly specific γ-secretase inhibitor. SH6 (AKT inhibitor III, Calbiochem) is a phosphatidylinositol analog that selectively inhibits the activation of AKT.

DNA Microarray Analysis.

Samples for microarray analysis were prepared and hybridized in Affymetrix Human U133 Plus 2.0 arrays according to the manufacturer's instructions. RNA was extracted from duplicate cultures of GSI-sensitive (ALL-SIL, CUTLL1, DND41, HPB-ALL, KOPTKI) and GSI-resistant (CCRF-CEM, MOLT3, P12 ICHIKAWA, PF382 and RPMI8402) T-ALL cell lines treated for 24 hours with vehicle (DMSO) or 500 nM CompE. Interarray intensity differences were normalized with Dchip 43.

Nearest-neighbor analysis was performed with signal-to-noise statistic (pclass 0 pclass 1)/(s class 0+s class 1) to establish the correlation of expression data with GSI-sensitive (class 0) and GSI-resistant (class 1) groups.

Proliferation and Cell Size Assays.

Changes in cell size were monitored by flow cytometry after NOTCH1 inactivation by GSI treatment (CompE 100 nM) and upon lentiviral shRNA knock-down of NOTCH1. Cell growth ratios were determined by a colorimetric assay using the Cell Proliferation Kit I (MTT) (Roche) in cells treated with inhibitors or vehicle treated controls.

Retroviral and Lentiviral Constructs and Viral Production.

Retroviral particles driving the expression of EGFP (MIG) and myristoylated AKT (MIG MYR AKT) were generated. Homogeneous populations of cells were obtained by FACS sorting of GFP-positive cells after spin infection. Oligonucleotide sequences for shRNAs targeting NOTCH1, PTEN, or the luciferase gene were cloned in the pLKO-puro and pGK-GFP lentiviral vectors (Dana-Farber Cancer Institute, Boston, Mass.). Lentivirus production and infections were performed using standard procedures.

Western Blot Analysis.

Antibodies against activated NOTCH1 (Val1744, Cell Signaling); PTEN (clone 6H2.1, Cascade Biosciences), phospho-AKT (Ser473), AKT (Cell Signaling), LC3, and α-tubulin (TU-02, Santa Cruz Biotechnology) were used according to standard procedures. PTEN immunostaining of formalin-fixed paraffin-embedded tissue sections was performed after heat-induced epitope retrieval in a microwave in citrate buffer (pH6.0). PTEN antibody (Zymed) was used at a 1:50 dilution. Slides were incubated at room temperature overnight before antigen detection using a Ventana automated staining platform (Ventana) and diaminobenzidine (DAB) detection.

PTEN Mutational Analysis.

PTEN transcripts were amplified from RNA extracted from cryopreserved lymphoblast samples provided by Dana-Farber Cancer Institute, St. Jude Children's Research Hospital, the Pediatric Oncology Group, and the Hospital for Sick Children. The transcripts were analyzed by direct bidirectional DNA sequencing. Analysis of PTEN exons 1-9 in additional diagnostic DNA samples and in paired diagnostic and relapse DNA samples from T-ALL patients enrolled in AIEOP-BFM Study Group protocols was performed by SURVEYOR digestion of DNA heteroduplexes, and the Transgenomic WAVE Nucleic Acid High Sensitivity Fragment Analysis system (WAVE HS; Transgenomic, Inc., Cambridge, Mass.), and verified by DNA sequencing.

Quantitative Real Time PCR.

Total RNA from T-ALL cell lines was extracted with RNAqueous kit (Ambion) following the manufacturer's instructions. cDNA was generated with the ThermoScript RT-PCR system (Invitrogen) and analyzed by quantitative real-time PCR (SYBR Green RT-PCR Core Reagents kit and the 7300 Real-Time PCR System, both from Applied Biosystems). Similar procedures were used to analyze RNA from DN3 cells purified from OP9 cocultures: Trizol method (Invitrogen) for RNA isolation, Omniscript RT kit (Qiagen) for cDNA synthesis and QuantiTect SYBR Green PCR kit (Qiagen) and the Applied Biosystems Sequence Detection System 7000 for PCR. Relative expression levels were based on GAPDH and β-actin as reference controls.

OP9 Cultures and Expression Analysis of DN3 Cells.

OP9-DL1 and OP9-control cells were generated from the OP9 bone marrow stromal cell line and maintained. Fetal liver (FL) was harvested from timed-pregnant Rag2−/− females on day 14 or 15 of gestation and single-cell suspensions were generated by disruption through a 40 μm nylon mesh using a syringe plunger. CD24Io/-FL cells, enriched for hematopoietic progenitor cells, were obtained by CD24 antibody and complement mediated lysis, and subsequently cultured with OP9-DL1 cell monolayers for T lineage differentiation. All cultures were supplemented with 1 ng/mL mouse IL-7 and 5 ng/mL human recombinant Flt-3 ligand (hrFlt3L; Peprotech). CD44-CD25+ GFPDN3 cells were purified by cell sorting from day 7 cultures, and further cultured for 1-2 days with either OP9-DL1 or OP9-control cells in the presence of cytokines, as above. CD45+ GFP−DN3 cells were sort purified from cocultures to exclude OP9 cells prior to quantitative real-time PCR analysis.

Metabolic Assays.

Glucose uptake and oxidation were analyzed in T-ALL cells treated with GSI (CompE 100 nM for 96 hours) or vehicle only (DMSO) controls. Briefly, cells (2×10⁶ per ml) were preincubated in serum-free RPMI medium for 45 minutes, washed and incubated for additional 45 minutes in 1 ml of serum/glucose-free RPMI medium containing glucose tracers. For glucose uptake, cells were incubated with 0.1 mM (2 μCi/ml) 2-[3H]-deoxy-glucose, then washed in cold PBS and solubilized in 0.1% SDS and analyzed by scintillation counting. For glucose oxidation, cells were incubated with 0.1 mM (2 μCi/ml) [U-¹⁴C]-glucose. At the end of the incubation period, cellular metabolism was blocked by the addition of perchloric acid. Glucose oxidation was measured as the amount of ¹⁴CO₂ captured in glass fiber filters previously soaked in 5% KOH. Glycolysis inhibition assays were performed with 500 μM 2-deoxy-glucose (Sigma) in cells growing in RPMI 1640 media supplemented with 10% fetal bovine serum.

Chip-On-Chip and Quantitative ChIP Analysis.

NOTCH1 (Val1744 antibody, Cell Signaling Technologies), HES1 (H-140, Santa Cruz Biotechnology) and MYC (N262 antibody, Santa Cruz Biotechnology) immunoprecipitates and control genomic DNA of HPB-ALL cells were differentially labeled with Cy3 and Cy5 and hybridized to the Agilent Proximal Promoter Arrays following standard procedures. Analysis and visualization of binding ratios for probes located in the PTEN proximal promoter were performed with Chip Analytics 1.1 software (Agilent Technologies) and the UCSC Genome Browser. Quantitative ChIP enrichment analysis of PTEN promoter sequences (−1492 to −1343; 612 to −445, and +118 to +278 from the transcription initiation site) in control genomic DNA (used as reference), and in chromatin immunoprecipitates performed with antibodies against HES1 (H-140, Santa Cruz Biotechnology), MYC (N262, Santa Cruz Biotechnology) and IgG (negative control) by real-time PCR was performed using β-actin genomic sequences levels as loading control.

PTEN-Luciferase Reporter Assays.

293T cells were transfected with a PTEN-luciferase reporter construct (pGL3 PTEN HindIII-NotI) and plasmids driving expression of HES1 (pEp7 HA-HES1) and/or c-MYC (pCMV MYC-FLAG) together with the pRL-CMV Renillaluciferase expression plasmid. PTEN reporter activity and Renilla luciferase levels (normalization control) were analyzed 48 hours after transfection with the Dual-Luciferase Reporter Assay kit (Promega).

Overexpression of Akt1 Gene in Drosophila.

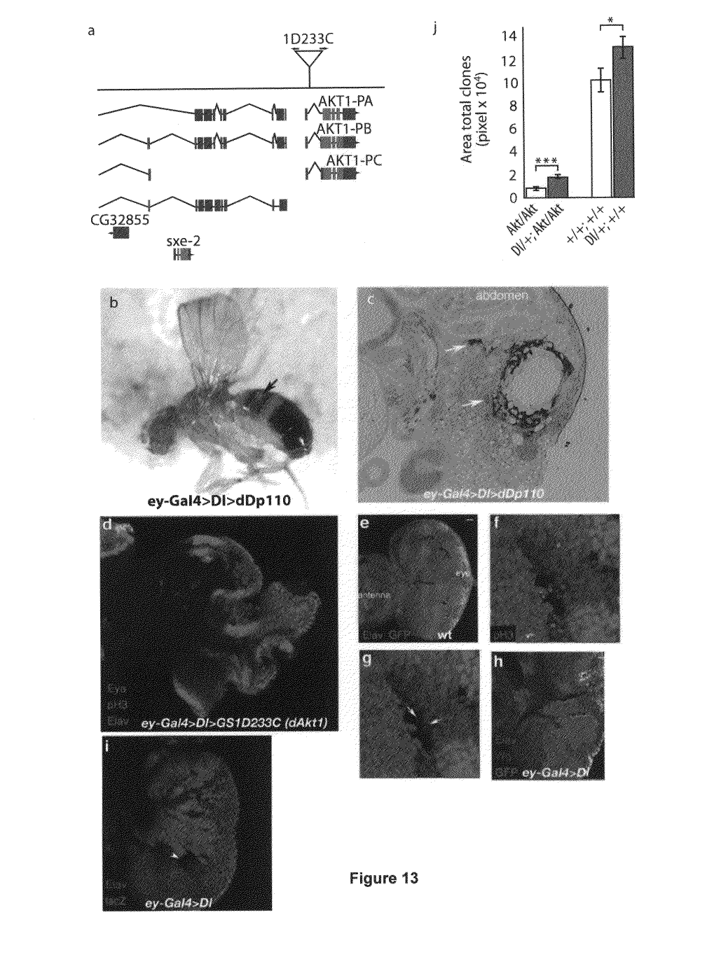

The Gene Search (GS) line 1D233C was isolated in a gain-of-expression genetic screen aimed at identifying genes that interact with the NOTCH pathway and that influence growth and tumorigenesis. Genomic DNA flanking the P-element insertion in the GS1D233C was recovered by inversed PCR (http://www.fruitfly.org/about/methods) and sequenced. A BLAST search with each sequence produced perfect matches to Akt1 gene at the interval 89B6 (chromosome 3R position 11925510-1111925511) (FIG. 13(a)). The GS P-element lines allow Gal4-dependent inducible expression of sequences flanking the insertion site of the GS element in both directions. Polymerase chain reaction with reverse transcriptase (RT-PCR) confirmed that transcription of the Akt1 gene is induced by the GS1D233 in response to Gal4 activation (data not shown).

PTEN Mutations in GSI Resistant T-ALL Cells

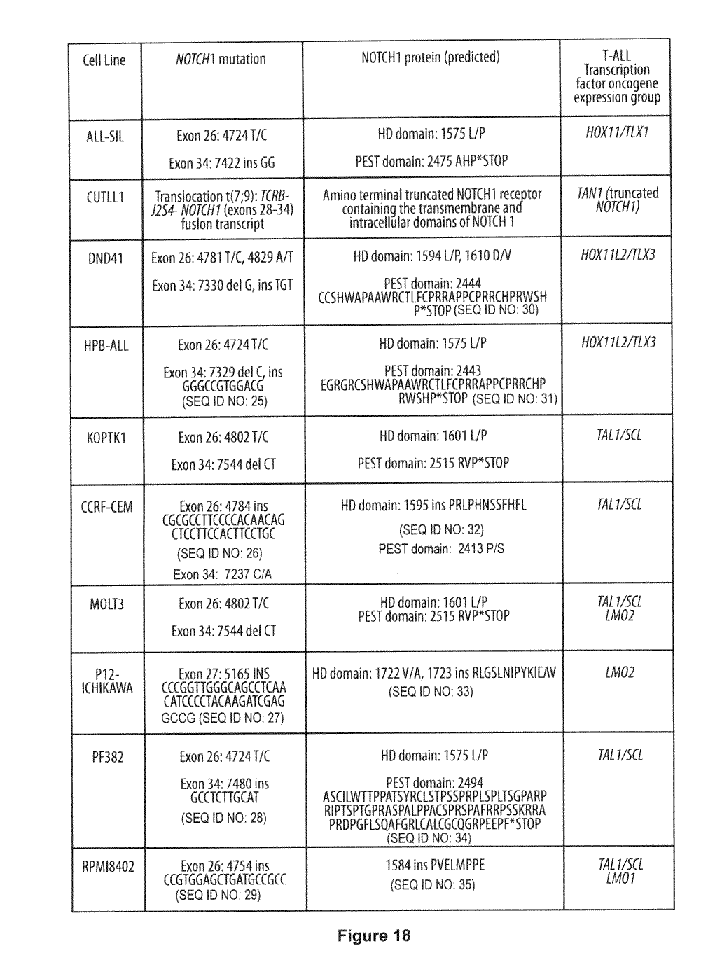

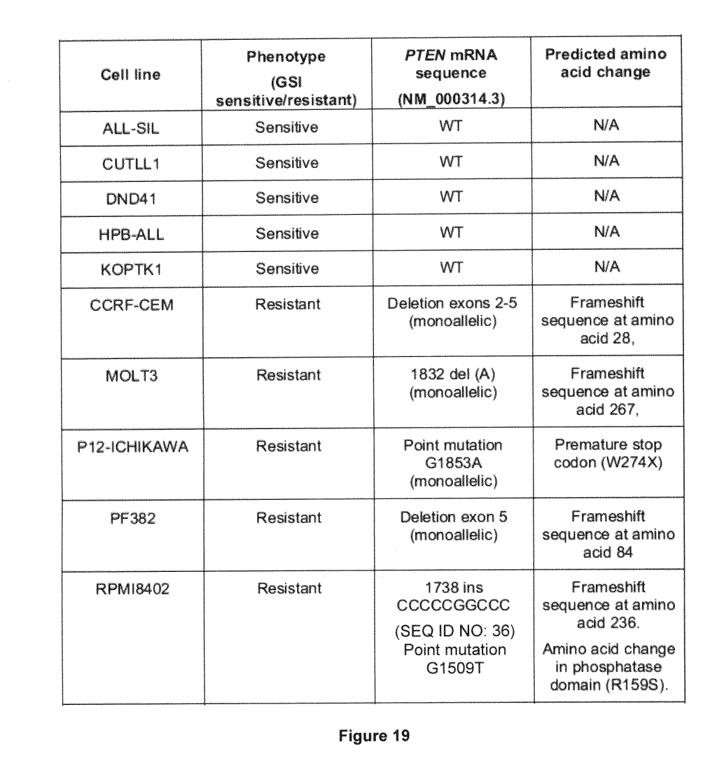

To elucidate the mechanism of resistance to GSIs in T-ALL, we tested the ability of a well-characterized GSI, Compound E (CompE), to inhibit NOTCH1 processing and NOTCH1 signaling in a panel of T-ALL cell lines harboring prototypical activating mutations in NOTCH1 (FIG. 18) previously characterized for their response to this GSI. Both GSI-sensitive (ALL-SIL, CUTLL1, DND41, HPB-ALL and KOPTK1) and GSI-resistant (CCRF-CEM, P12-ICHIKAWA, PF382, MOLT3 and RPMI8402) cell lines showed NOTCH1 inhibition when treated with CompE (FIGS. 18 and 19), leading us to consider that resistance to GSI action may be mediated by molecular abnormalities in signaling pathways that promote cell growth downstream of NOTCH1.

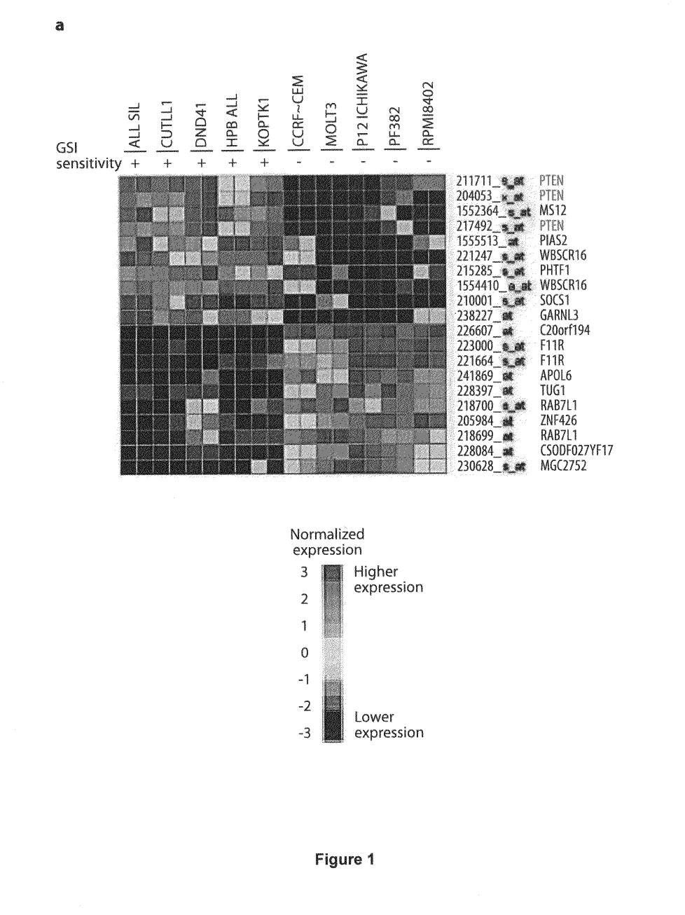

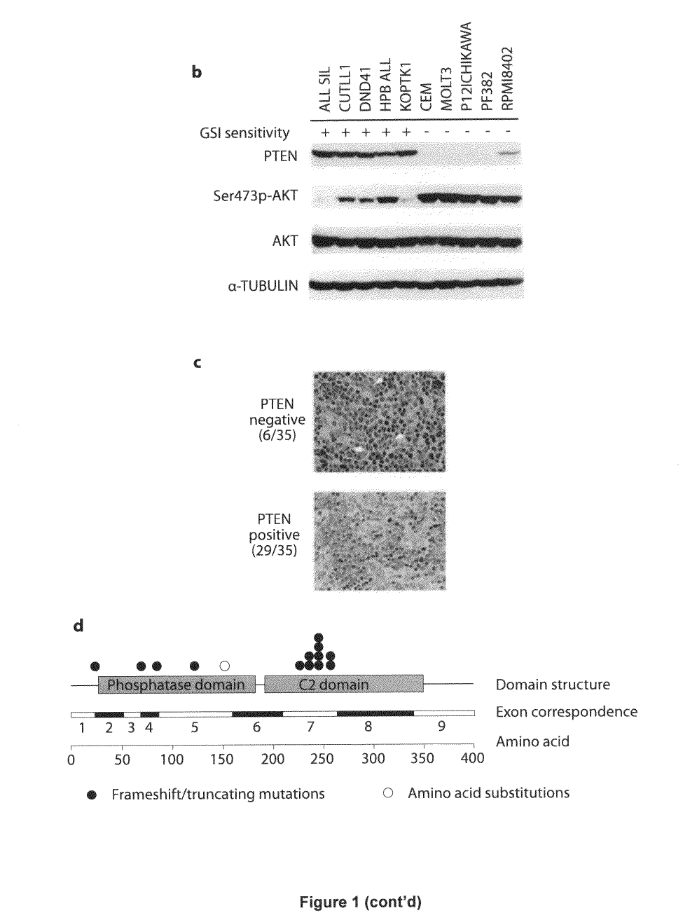

To test this hypothesis, we analyzed this panel of well characterized GSI-sensitive and GSI-resistant cell lines with oligonucleotide microarrays to identify differentially expressed genes associated with GSI sensitivity or resistance. Nearest-neighbor analysis using the signal-to-noise statistic identified PTEN, which encodes a key tumor suppressor that inhibits the PI3K-AKT signaling pathway, as the gene most consistently downregulated in GSI-resistant cell lines (FIG. 1(a)). Western blot analysis showed readily detectable PTEN protein in all GSI-sensitive T-ALL cells, but total absence or a marked decrease in PTEN levels in all five GSI-resistant cell lines analyzed (FIG. 1(b)).

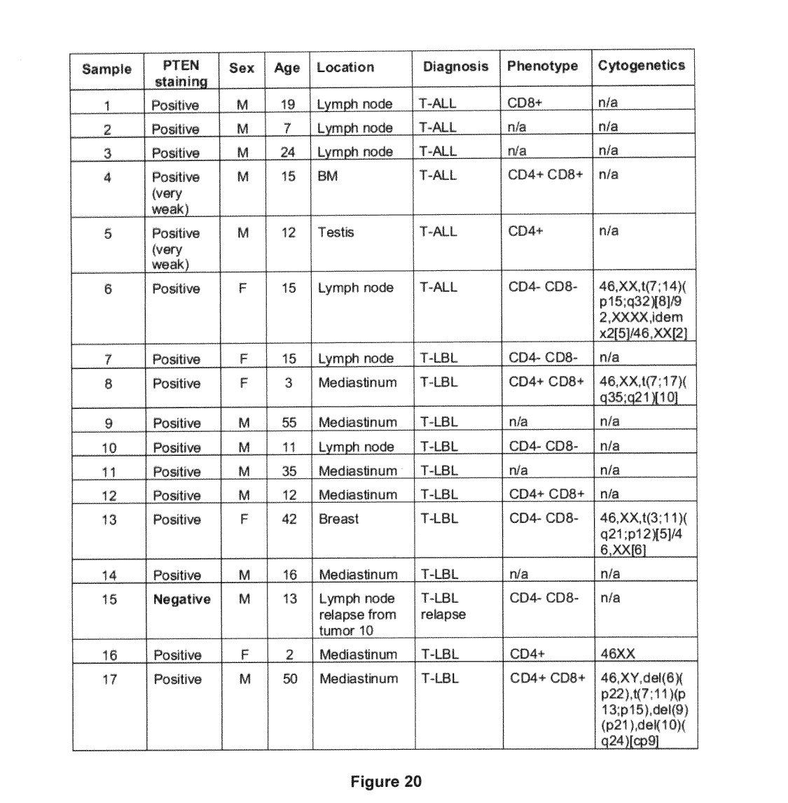

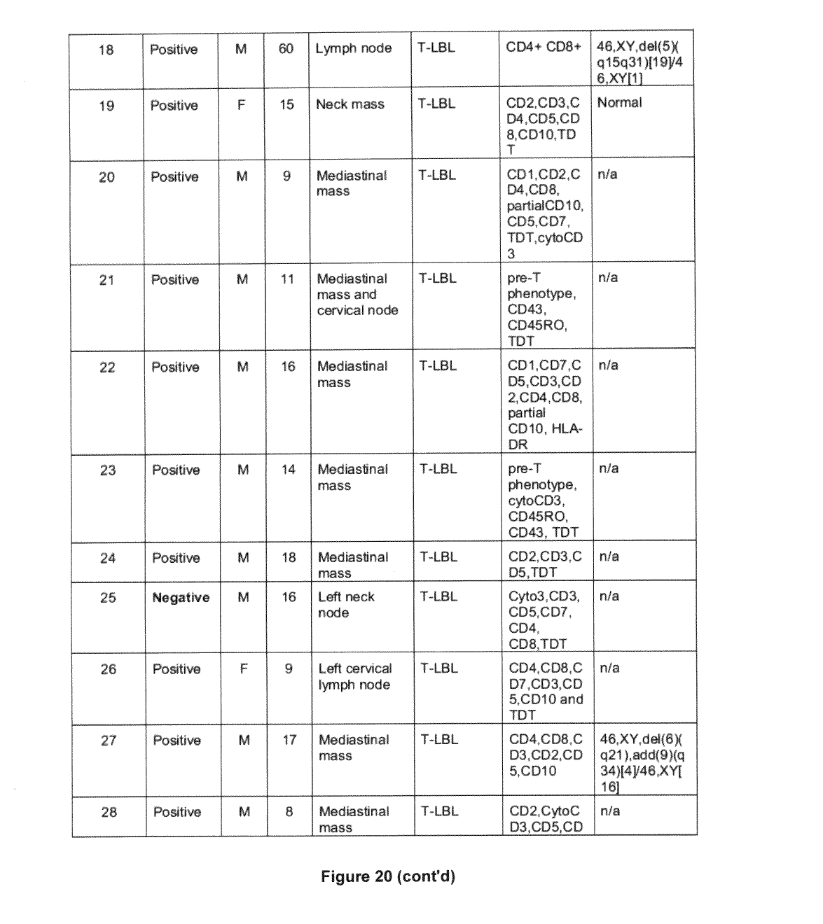

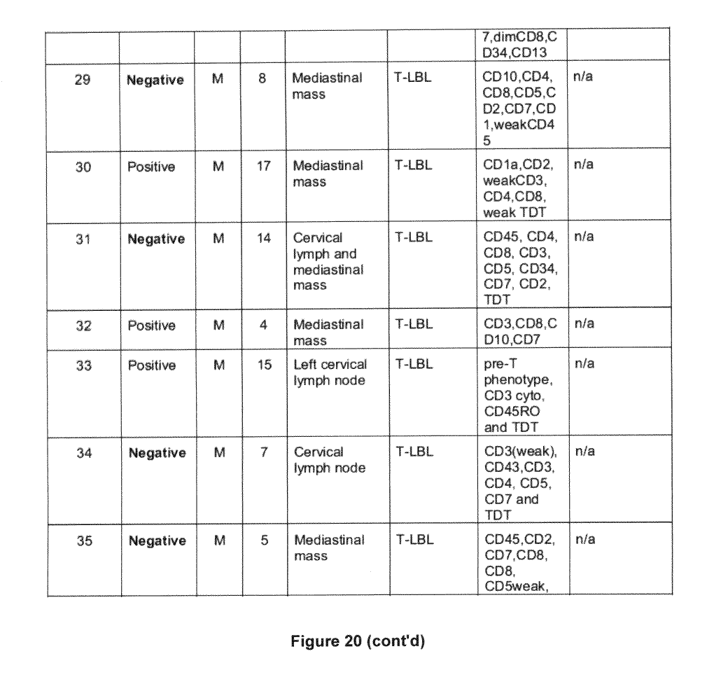

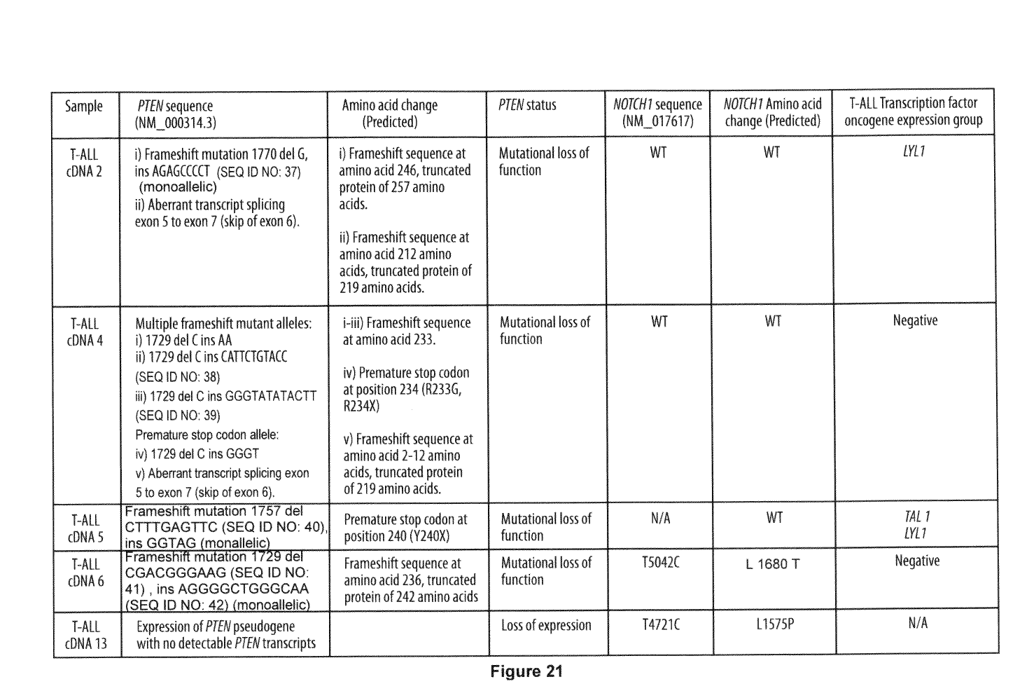

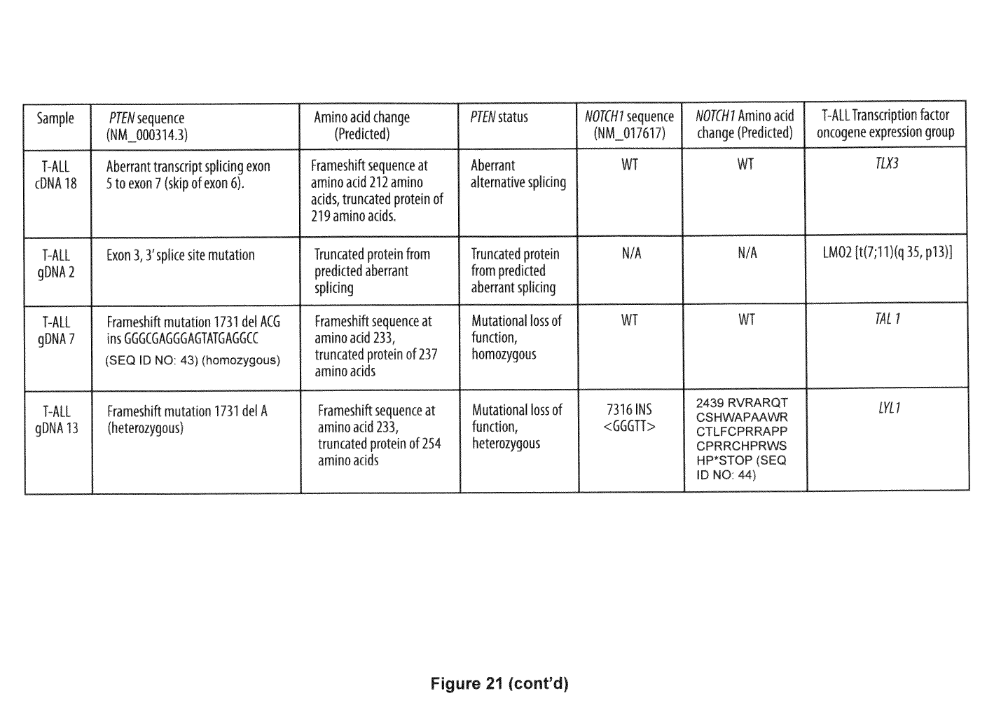

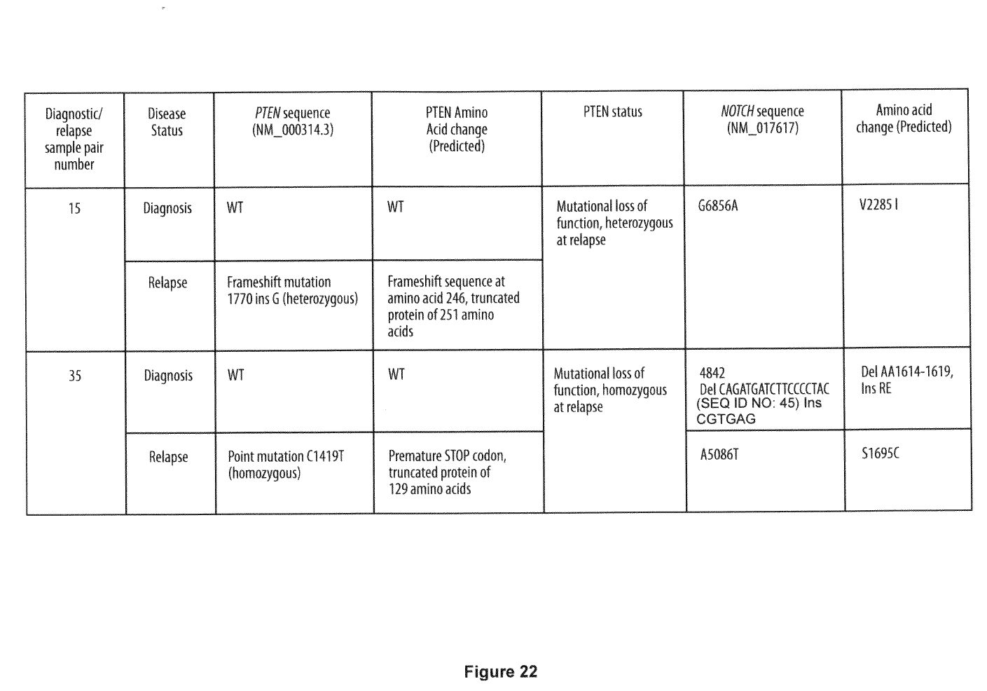

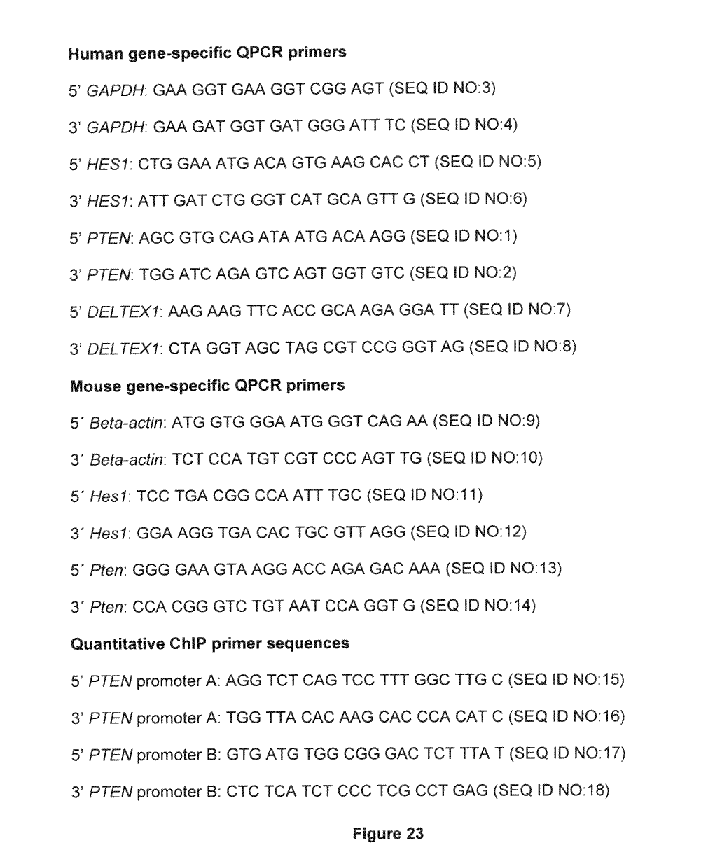

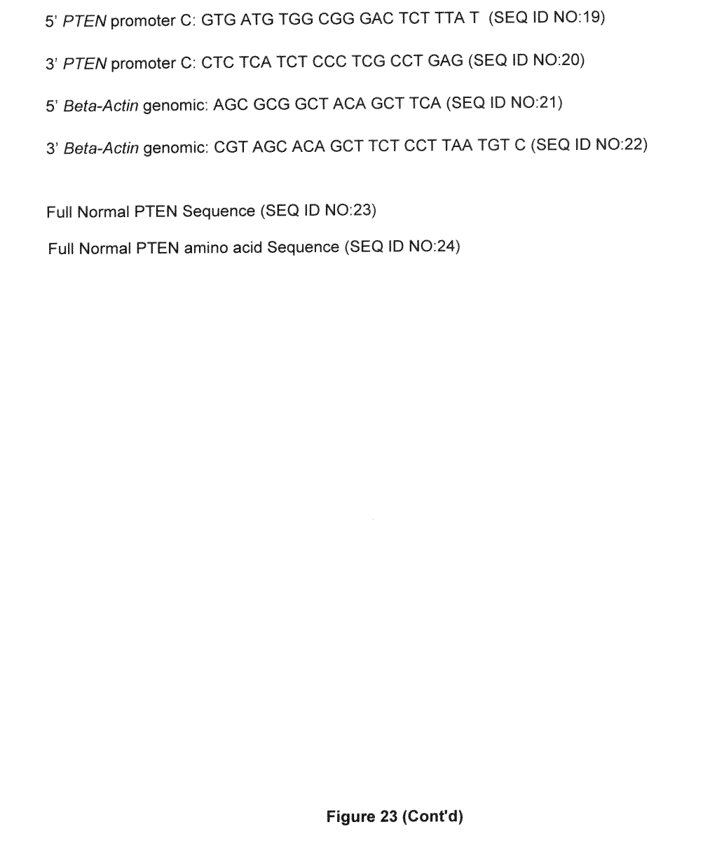

Further analysis demonstrated that each of the five GSI-resistant cell lines harbored mutations in PTEN, while the five GSI-sensitive cell lines expressed normal PTEN transcripts (FIG. 19). PTEN mutations associated with GSI resistance were typically homozygous and generated truncated PTEN protein products due to the presence of premature stop codons. Only RPMI8402 cells contained both a truncating mutation in PTEN and a missense mutation (R159S) in the phosphatase domain in the other allele, consistent with the presence of detectable PTEN protein in this cell line and variable response to GSI. PTEN mutations are frequent in solid tumors and loss of PTEN has been shown to promote the self renewal of leukemic stem cells. Furthermore, a role of PTEN deficiency in the pathogenesis of T-cell tumors has been proposed based on the analysis of PTEN knock-out mice. However, PTEN mutations have only been reported sporadically in human leukemias and lymphomas. Thus, to determine whether our discovery of PTEN loss in GSI-resistant cell lines might also be relevant to primary human cancers, we examined the status of PTEN in T-ALL clinical samples. We found loss of the PTEN protein by immunohistochemistry in 6 of 35 (17%) T-ALL samples, including one relapsed sample in a lymphoma case that was PTEN positive at diagnosis (FIG. 1(c) and FIG. 20). Sequence analysis demonstrated mutations in PTEN in 9 of 111 (8%) T-ALL cases at diagnosis (FIG. 1(d)). In addition, one case expressed the PTEN pseudogene with no expression of normal PTEN transcripts (FIG. 21). Subsequent analysis of 35 paired samples of DNA from T-ALL lymphoblasts collected at diagnosis and at relapse demonstrated two additional PTEN mutant cases in which loss of this tumor suppressor gene occurred during disease progression (FIG. 22). Thus, PTEN mutations and loss of PTEN protein expression are highly frequent in T-ALL cell lines, occur in a subset of human T-cell leukemias and lymphomas at diagnosis, and can be found also as a secondary event during disease progression.

Aberrant AKT Signaling Induces Resistance to GSI.

As a critical regulator of the PI3K-AKT signal transduction pathway, PTEN controls multiple cellular responses, including metabolic regulation and cell growth and survival. Activation of phosphatidylinositol 3-kinase (PI3K) by extracellular stimuli generates phosphatidylinositol triphosphate (PIP3) in the plasma membrane, which recruits the AKT kinase to the membrane, where it is phosphorylated and activated by phosphatidyl inositol-dependent kinase-1 (PDK1). Upon its activation, AKT triggers the phosphorylation of numerous protein targets, such as the mTOR kinase, and influences multiple cellular processes including cell growth and proliferation. The PTEN gene encodes a lipid phosphatase that is responsible for PIP3 dephosphorylation and clearance and required to switch off AKT activation. To pursue this putative link between PTEN and the PI3K-AKT pathway in T-ALL, we examined the levels of AKT phosphorylation in our panel of GSI-sensitive/PTEN-positive and GSI-resistant/PTEN-null cell lines. Western blot analysis showed that p-AKT (Ser473) levels were low and inversely correlated with PTEN expression in GSI-sensitive/PTEN-positive T-ALL cells (FIG. 1(b)). By contrast, GSI-resistant/PTEN-null T-ALL samples showed high levels of AKT phosphorylation, indicative of constitutive activation of the PI3K-AKT signaling pathway. To test the prediction that unrestrained activation of the PI3K-AKT signaling pathway plays a causative role in resistance to GSIs, we expressed a constitutively active myristoylated form of AKT (MYR-AKT) in the GSI-sensitive CUTLL1 cell line.

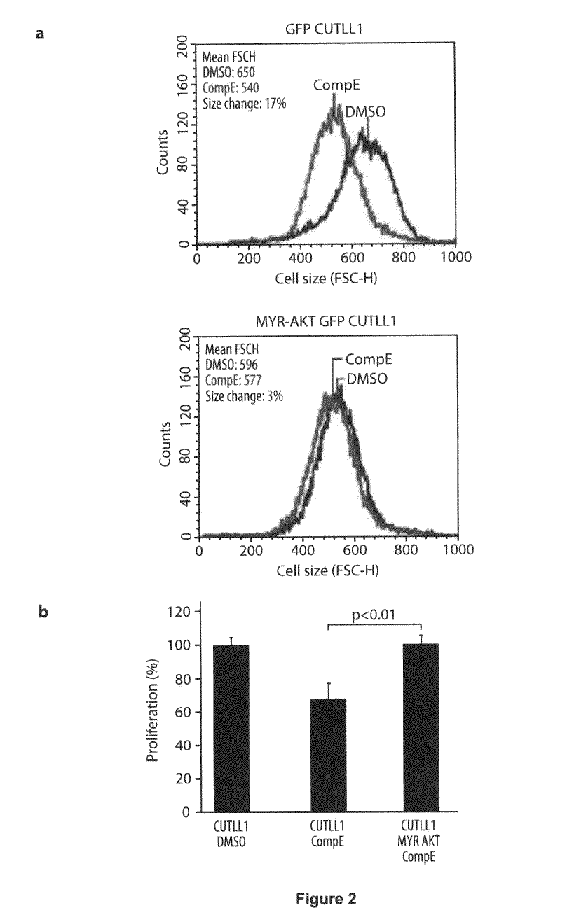

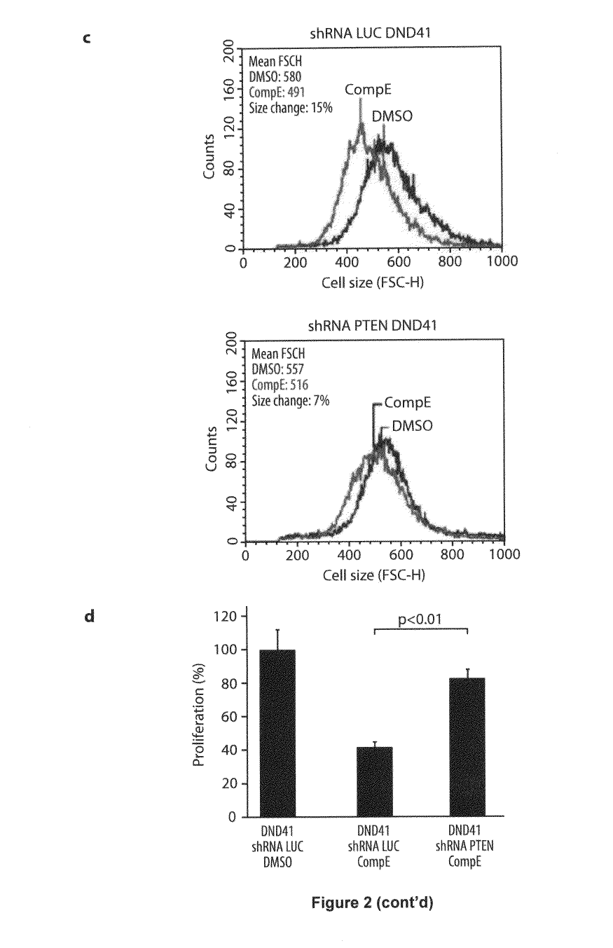

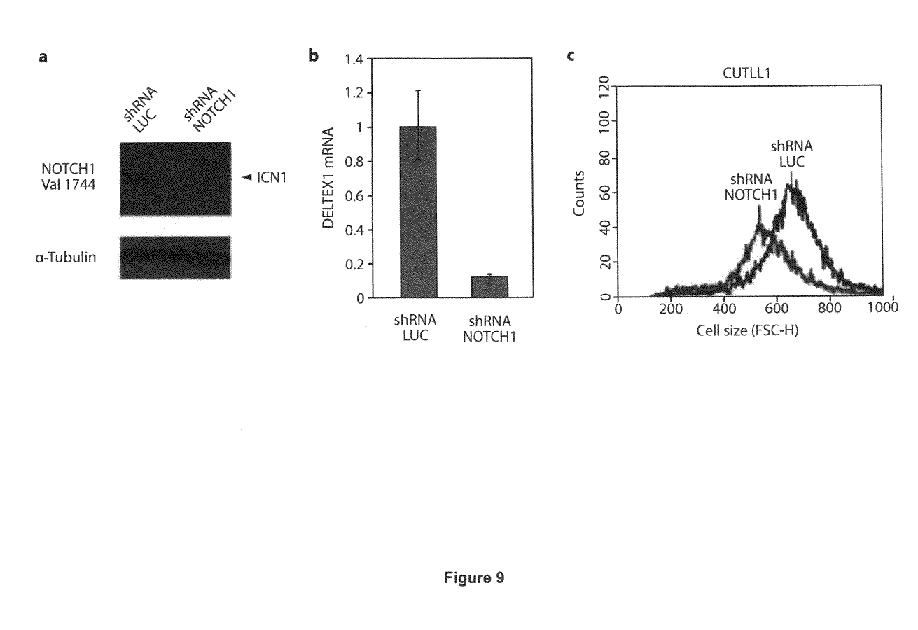

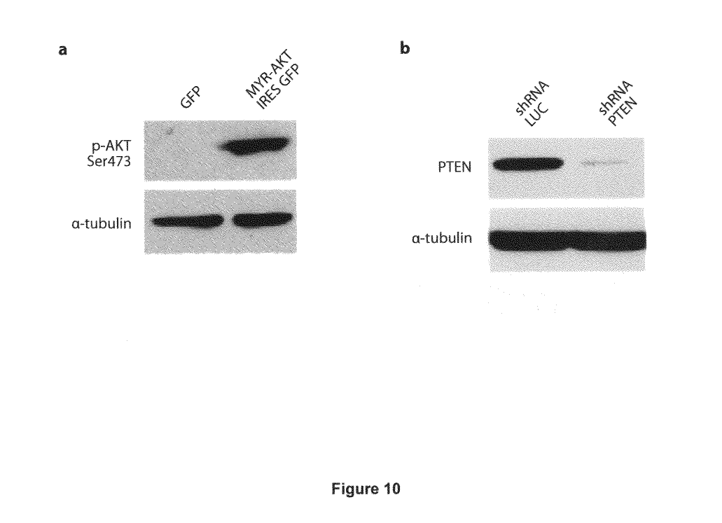

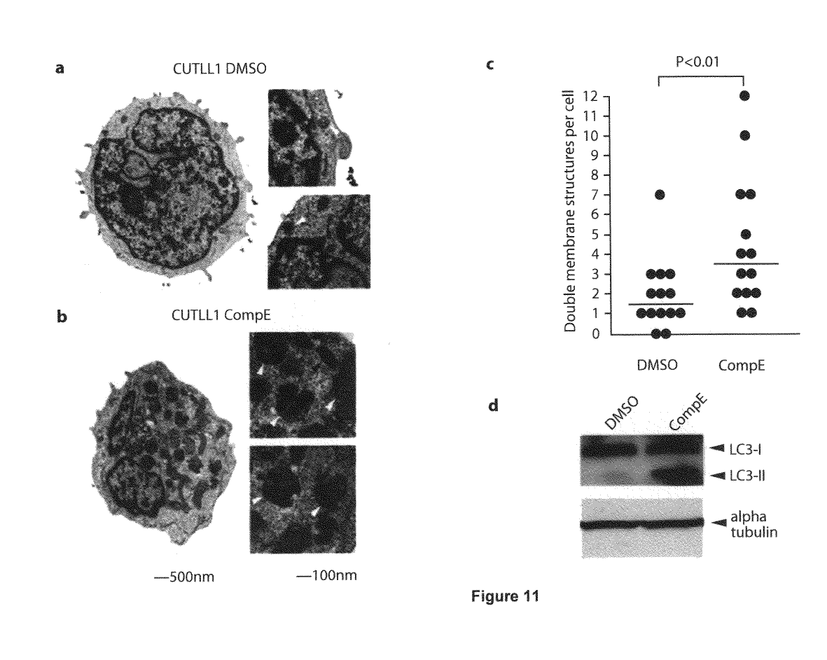

Inhibition of NOTCH1 signaling by GSI treatment or NOTCH1 shRNA knock-down typically impaired the growth of CUTLL1 cells (FIG. 2(a) and FIG. 9), whereas constitutively high levels of p-AKT (Ser473) (FIG. 10) induced by enforced expression of MYR-AKT was sufficient to rescue these cells from the growth inhibitory effects of NOTCH1 inhibition with GSI (FIG. 2(a)-(b)). Similarly, shRNA knock-down of PTEN in DND41 cells (FIG. 10) blocked the cell growth inhibitory effects of CompE in this GSI-sensitive/PTEN-positive cell line (FIG. 2(c)-(d)). Together, these results indicate that aberrant activation of the PI3K-AKT signaling pathway induces resistance to NOTCH1 inhibition in T-ALL cells.

NOTCH1 Regulates PTEN and the PI3K-AKT Pathway.

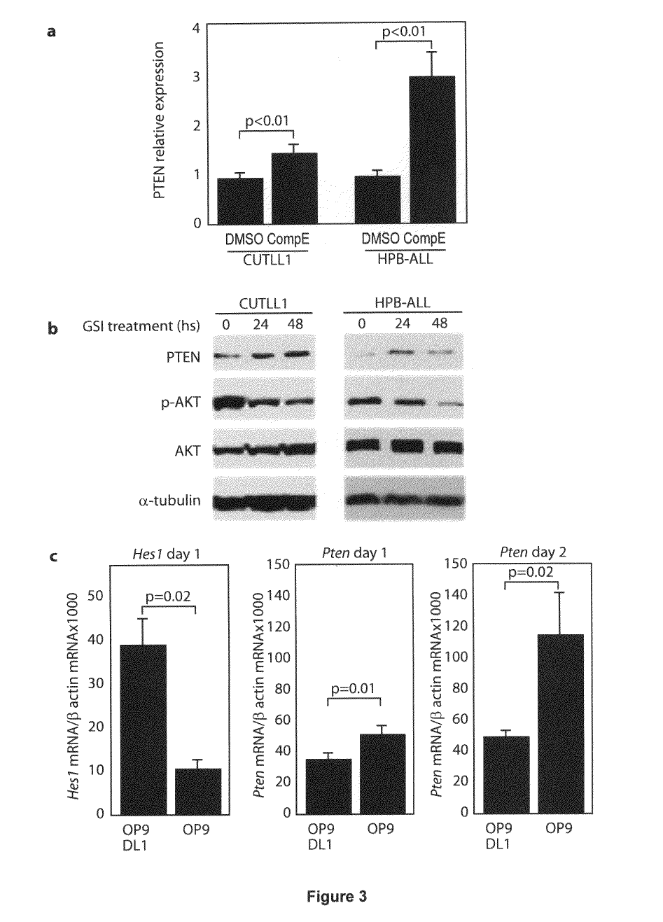

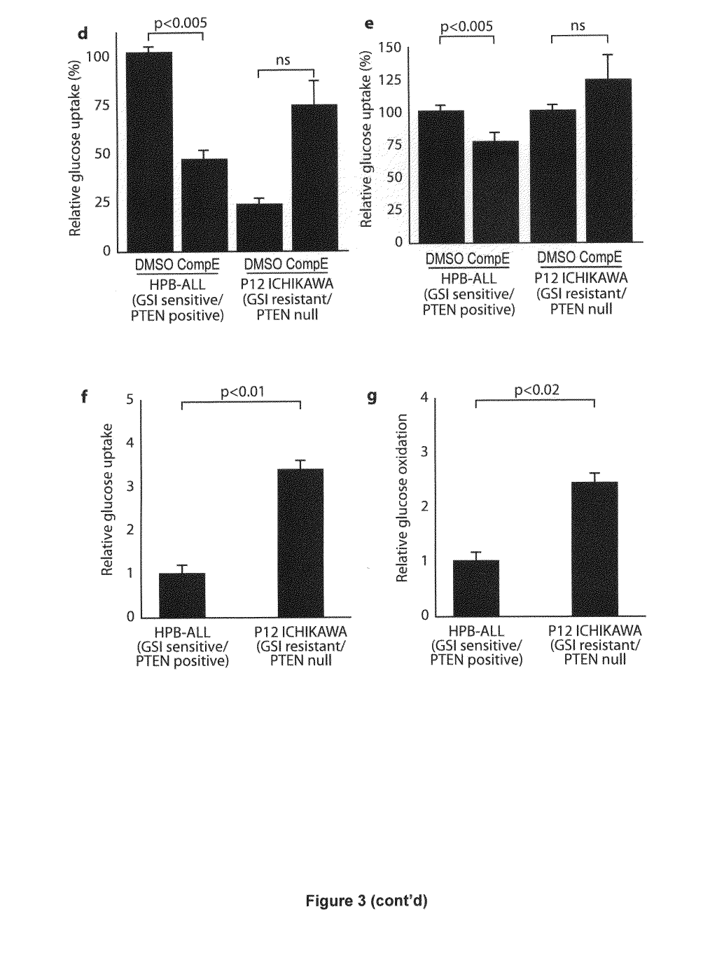

The close association between the presence of PTEN mutations and GSI resistance in T-ALL prompted us to ask whether PTEN might be functionally linked to NOTCH1 signaling. Analysis of the transcriptional responses of GSI-sensitive/PTEN-positive cells to NOTCH1 inhibition demonstrated significant upregulation of PTEN expression (FIG. 3(a)), with consequently higher PTEN protein levels, and gradual inhibition of the PI3K-AKT pathway, as judged from decreased Ser473 phosphorylation on AKT (FIG. 3(b)). Ligand-mediated activation of NOTCH signaling during T-cell development is required to maintain cell growth and glucose metabolism at the time of T-cell receptor β-chain selection and has been associated with increased AKT phosphorylation. Yet, the mechanism responsible for PI3K-AKT upregulation downstream of NOTCH activation during normal thymocyte development remains unknown. We therefore hypothesized that transcriptional downregulation of PTEN downstream of NOTCH1 could mediate the upregulation of the PI3K-AKT signaling pathway, not only in T-ALL cells but also in developing thymocytes. To test this hypothesis, we analyzed the effects of withdrawing NOTCH1 signals, driven by the NOTCH1 ligand Delta-like 1, from T-cell precursors.

Immature CD4-CD8-double-negative 3 (DN3) thymocytes were generated by coculture of hemopoietic progenitors from Rag−/− mice with stromal cells expressing the NOTCH ligand Delta-like 1 (OP9-DLI). Purified DN3 cells were subsequently cultured in the presence of continuous NOTCH1 signaling, by coculture with OP9-DL1 cells, or they were deprived of Delta-like 1 stimulation of NOTCH1, by coculture with regular OP9 stromal cells devoid of this ligand. Loss of NOTCH1 signaling in the DN3 thymocytes cultured in OP9 cells induced marked downregulation of the NOTCH1 target gene Hes1 at day 1 and progressive upregulation of PTEN transcript levels, compared to DN3 cells maintained in culture with OP9-DL1 cells (FIG. 3(c)). These results demonstrate that the regulation of PTEN expression downstream of NOTCH1 is not limited to human T-ALL cells harboring oncogenic NOTCH1 alleles, as it is also present in normal murine thymocytes upon activation of the wild type NOTCH1 receptor by the DL1 ligand.

Detailed phenotypic analysis of cellular responses to NOTCH1 inhibition in T-ALL showed that blocking NOTCH1 signaling with shRNA knock-down or GSI treatment in PTEN-positive T-ALL cells, induced cellular responses typically associated with inhibition of the PI3K-AKT signaling pathway, such as decreased cell size (FIG. 9), reduced glucose metabolism (FIG. 3(d)-(e)) and increased autophagy (FIG. 11). Analysis of glucose use in GSI-sensitive/PTEN-positive HPB-ALL cells demonstrated significant reductions in glucose uptake and glucose oxidation upon NOTCH1 inhibition, and increased levels of glucose uptake and glucose oxidation which were not affected by GSI treatment in GSI-resistant/PTEN-null P12ICHIKAWA cells (FIG. 3(e)-(g)). These results suggest that a NOTCH1-PTEN-AKT regulatory axis mediates the physiologic upregulation of the PI3K-AKT signaling pathway during normal thymocyte development, while aberrant NOTCH1 signaling in T-ALL converts this developmental transcriptional network to a mechanism that promotes leukemic cell growth.

HES1 and MYC Mediate Regulation of PTEN Expression Downstream of NOTCH1.

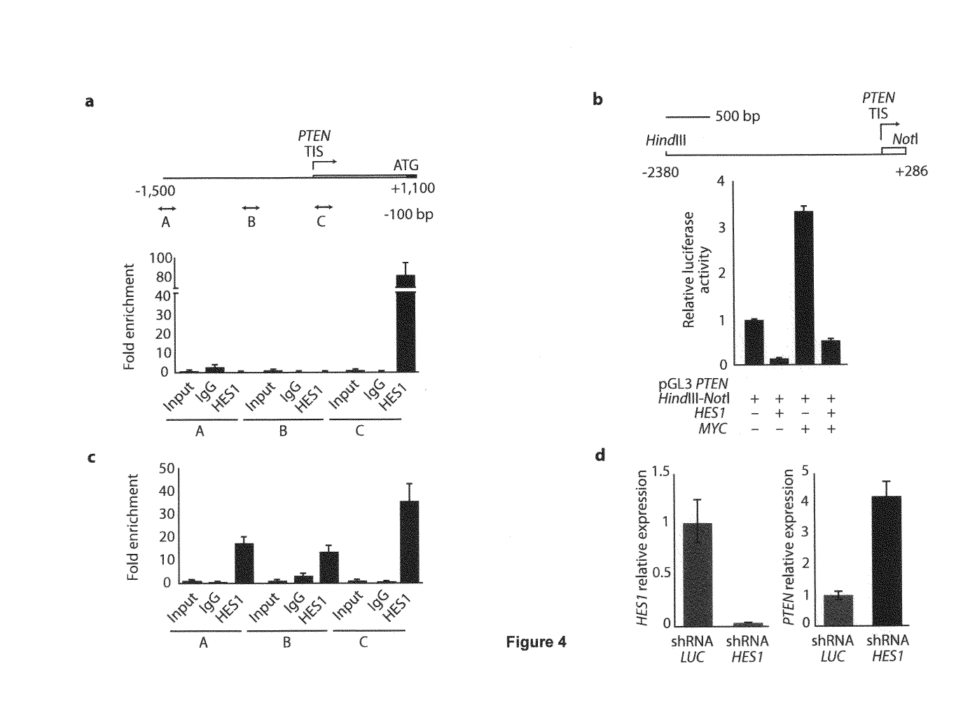

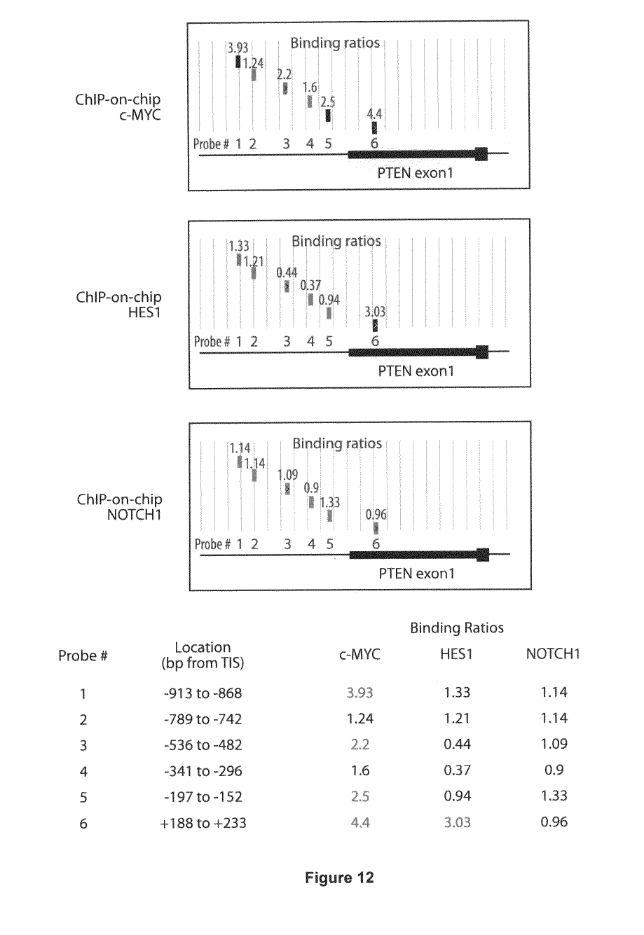

The inhibitory effect of NOTCH1 signaling on PTEN expression conflicts with the well-established role of NOTCH1 as transcriptional activator. Thus, we considered that inhibition of PTEN by NOTCH1 could be mediated by HES1 and MYC, two transcription factors directly controlled by NOTCH1. ChIP-on-chip analysis of promoter occupancy by HES1, MYC and NOTCH1 in the HPB-ALL leukemic cell line identified binding of both MYC and HES1 to regulatory sequences in the PTEN proximal promoter (FIG. 12); a result that was fully validated by quantitative ChIP assays (FIG. 4(a)-(b)). While HES1 bound in the vicinity of the PTEN transcription initiation site, MYC occupancy of the PTEN promoter comprised at least two regulatory regions located around positions −1,400 and −500 base pairs upstream of the PTEN gene, as well as the region occupied by HES1, 200 by downstream from the PTEN transcription initiation site. The functional significance of HES1 and MYC binding to the PTEN promoter was demonstrated in luciferase reporter assays, which showed that HES1 expression induced a 20-fold reduction in the activity of a 3-kb PTEN promoter construct, while MYC expression induced a 3-fold increase in luciferase expression over basal levels (FIG. 4(c)). Promoter batching analysis demonstrated redundancy of regulatory sequences occupied by MYC in the activity of PTEN reporter constructs and confirmed the dominant role of HES1 as negative regulator of the PTEN promoter (data not shown). Furthermore, shRNA knock down of HES1 induced transcriptional upregulation of PTEN transcript levels in T-ALL cells (FIG. 4(d)). These findings are in agreement with the analysis of Hes1 knock-out mice which showed an essential role of this transcriptional repressor downstream of NOTCH1 in promoting cell growth and proliferation during thymocyte development.

Conservation of the NOTCH-PTEN-AKT Regulatory Axis in Drosophila.

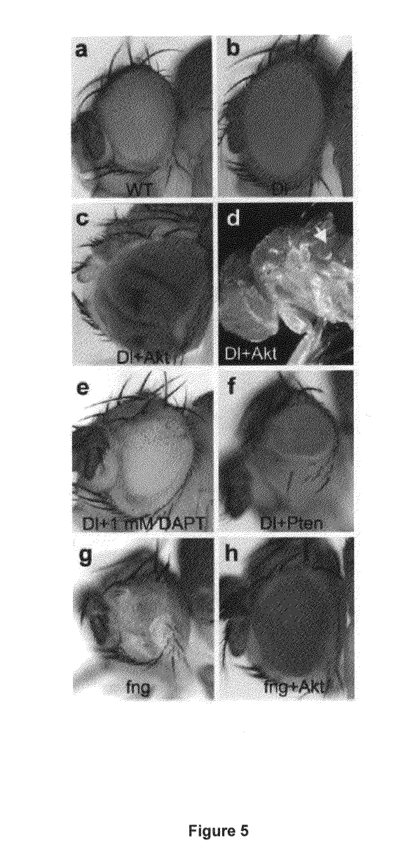

We have established a highly specific forward genetic screen in Drosophila to search for genes that functionally cooperate with the NOTCH pathway and that convert tissue overgrowths into tumors. The significance of the interaction between NOTCH and PI3K-AKT signaling in promoting oncogenic cell growth and proliferation was further reinforced by the independent identification of a close relationship among NOTCH, PTEN, and Akt during tumorigenesis in this model (FIGS. 5, 13-15).

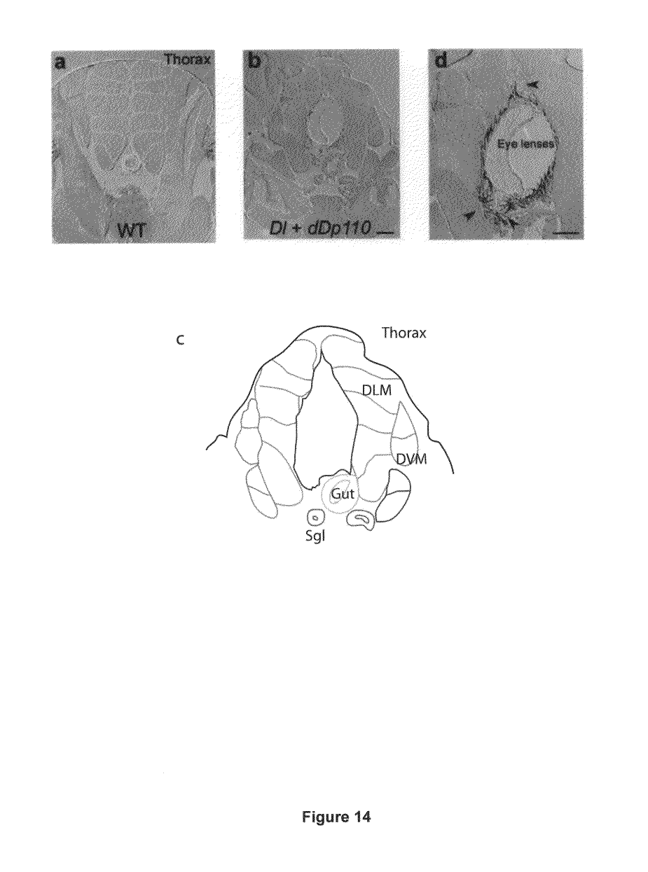

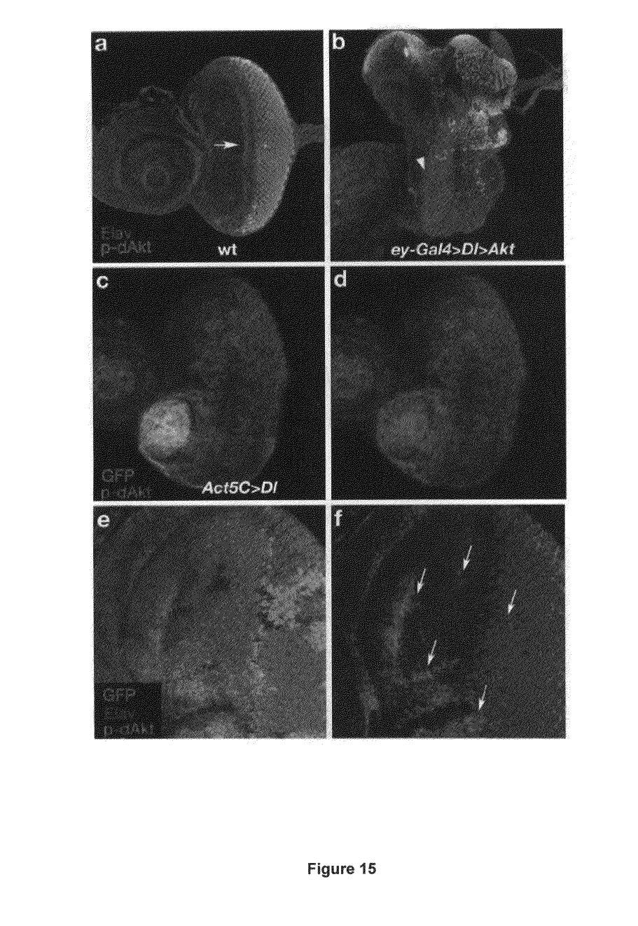

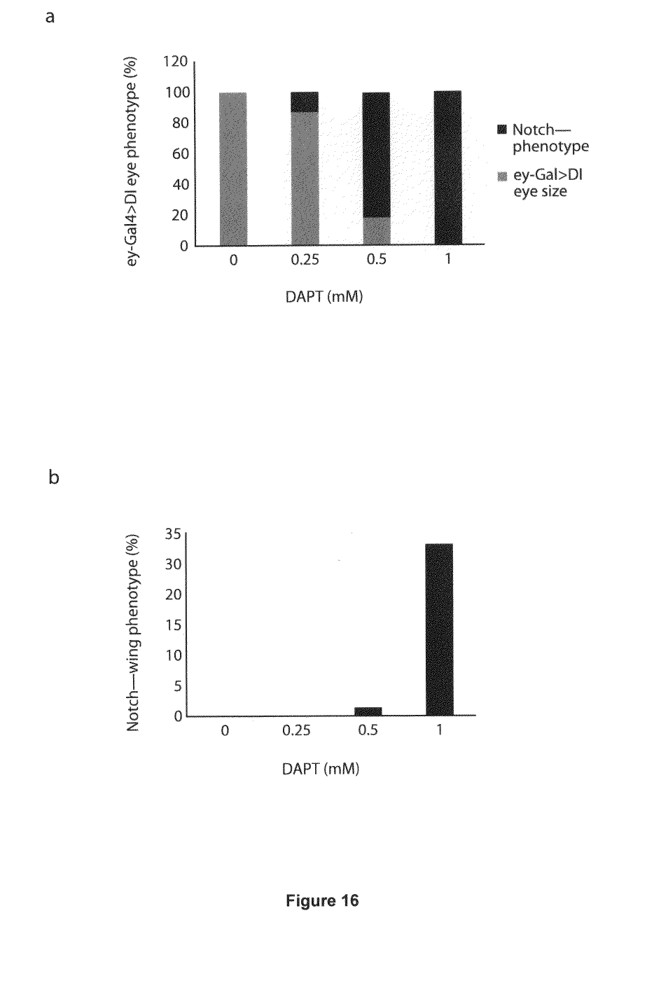

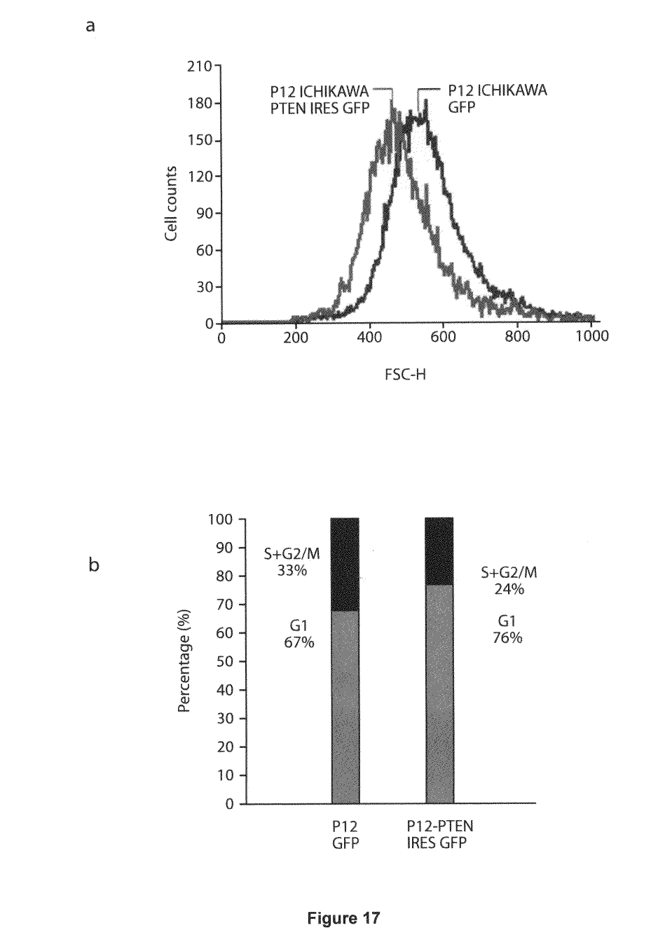

We used a Drosophila strain that overexpressed the NOTCH ligand Delta, which caused a ‘large eye’ phenotype due to overgrowth (FIG. 5(b)), coupled to the Gene Search method to systematically generate ‘gain-of-expression’ mutations (FIG. 5(a)-(b)). In this screen we identified a GS line 1D233C that enforced expression of the Akt1 gene (FIG. 13(a)), and that when coupled to Delta overexpression produced massive overgrowths and tumors (FIG. 5(c)-(d) and FIG. 13(d)), leading to disseminated eye-derived secondary growths inside the thorax and abdomen in about 8% of the flies (FIG. 5(d)). Eye overgrowths and secondary eye growths were also observed, albeit with less occurrence, in animals with Delta overexpression combined with a transgene of the Drosophila Phosphatidylinositol 3-OH kinase 92E (Dp110) (FIG. 13(b)-(c) and FIG. 14). Importantly, simply overexpression of Akt1 alone did not result in eye overgrowth or tumor development, neither resulted in a noticeable increase in Akt phosphorylation (data not shown). In contrast, phospho Akt (pAkt) was found to be slightly increased in the eye imaginal discs overexpressing Delta and more markedly augmented in the eye imaginal discs that overexpressed Delta and Akt1 (FIG. 15(a)-(d)), suggesting a functional relationship between NOTCH signaling and Akt pathway in growth control. Pharmacological inhibition of NOTCH receptor proteolysis by a GSI (DAPT) prevented eye growth induced by Delta overexpression and resulted in a small-eye defect (FIG. 5(e) and FIG. 16), recapitulating the defective growth phenotype of PTEN-positive T-ALL cells treated with GSI. Importantly, a similar small-eye phenotype was induced upon overexpression of PTEN, indicating that inhibition of Akt phosphorylation can block the growth promoting effects of NOTCH hyperactivation (FIG. 5(f)). Moreover, loss of Akt1 (analyzed in genetic mosaics using an Akt1 null allele, FIG. 13(e)-(j)) also blocked Delta-induced eye overgrowth. Note that, in contrast to previous reports, it was found that inactivation of Akt1 does affect cell number (FIGS. 13(e)-(g) and (j)), suggesting that, as its mammalian counterpart, Drosophilia Akt1 positively regulates cell-cycle progression.

Consistently, the enforced expression of Akt1 by use of the GS1D233C (FIG. 13(a)), fully rescued the eye growth arrest caused by a reduction of NOTCH signaling due to generalized expression of the fringe (fng) gene (FIG. 5(g)-(h)), recapitulating the restoration of cell growth induced by constitutive active AKT expression in GSI-sensitive/PTEN-positive T-ALL cells treated with CompE, and the resistance to GSI treatment phenotype of PTEN-null T-ALL cell lines (FIGS. 1-2). Together, these results provide compelling evidence that Akt signaling is essential for eye growth induced by the Delta-NOTCH pathway, that activation of Akt1 restores growth upon NOTCH inhibition and that aberrant Akt1 activity synergizes with NOTCH hyperactivation to promote tumor development in vivo.

Secondary Oncogene Addiction in GSI-Resistant/PTEN-Null T-ALL Cells.

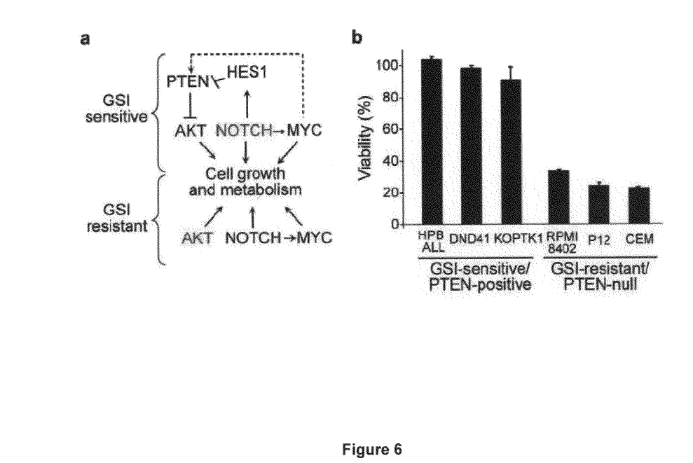

Based on our results showing that loss of PTEN in T-ALL cells may result in resistance to NOTCH1 inhibition, we hypothesized that GSI-resistance could occur at the expense of making PTEN-null T-ALL cells addicted to constitutive AKT signaling (FIG. 6(a)). Importantly, PTEN deficient tumors have been shown to be hypersensitive to inhibition of the P13K signaling pathway and reintroduction of PTEN in the GSI-resistant/PTEN-null Jurkat T-cell leukemia cell line has been shown to induce cell cycle arrest, decreased cell size and increased apoptosis. To test this hypothesis we treated GSI-sensitive/PTEN-positive and GSI-resistant/PTEN-null T-ALL cells with SH-6, a phosphatidylinositol analog inhibitor of AKT. Importantly, pharmacologic inhibition of AKT showed strong antileukemic effects in GSI-resistant/PTEN-null T-ALLs but not in GSI-sensitive/PTEN-positive cells (FIG. 6(b)). Similarly, retroviral expression of PTEN in GSI-resistant T-ALL cells induced a marked reduction in cell size and G1 cell cycle arrest (FIG. 17). Thus, aberrant activation of the PI3K-AKT pathway makes PTEN-null T-ALL cells dependent on high levels of AKT signaling for maintenance of the malignant phenotype in a clear example of secondary oncogene addiction.

The following examples demonstrate yet further aspects and embodiments of the present invention.

Mice. Rag2-deficient mice were bred and maintained in the animal facility of the Sunnybrook Research Institute under specific pathogen-free conditions. Animal procedures were approved by the Sunnybrook Research Institute Animal Care Committee (Toronto, Ontario, Canada).

Electron microscopy. Sample processing for transmission electron microscopy analysis of autophagy was performed using standard procedures. Double membrane structures, excluding mitochondria, were counted in sample preparations of an early-pass culture of CUTLL1 cells treated with DMSO or CompE (500 nM) for 6 days.

Drosophila husbandry. Fly stocks and genotypes used were: ey-Gal4, UAS-DI, UASPTEN, GS1D233C (Akt1, see below), UAS-Dp110 (PI3K), UAS-fng. Loss-of-function clones of Akt1 were induced in a gain-of-function Delta mutant larvae resulting from the cross between: yw;ey-Gal4 UAS-DI/CyO twist-GFP; FRT 82B arm-lacZ and ey-flp/Y; FRT 828 Akt1^{q}/TM6b. All fly stocks used (except GS1D233C) are described in http://flybase.bio.indiana.edu/. All flies were grown at 25° C. (except when indicated).

Drosophila DAPT treatments. The γ-secretase inhibitor DAPT prevents NOTCH processing, translocation and signaling in cell culture and induces developmental defects in Drosophila similar to those caused by genetic reduction of NOTCH signaling. Stock solutions of the compound in ethanol were added to 2.5 ml of distilled water and dried potato food were prepared. Flies carrying a Delta transgene (UAS-DI) and the eye-specific Gal4 driver, ey-Gal4 (hereafter, ey-Gal4>DI) were kept on standard fly food at 26° C. for 3 days (72 hours) and the progeny were collected and transferred to the vials containing the food mixed with DAPT and allowed to progress through development in the presence of DAPT until closure.

Mosaic analyses of Drosophila Akt1 loss-of-function clones. Eye imaginal discs carrying loss-of-function clones of Akt1 (null allele of Akt1, Akt1q 6) in an ey-Gal4>DI and control (wild type) background were produced by mitotic recombination using the hsp70-Flp and the ey-FLP/FRT techniques.

The genotypes in FIG. 13 are: hsp70-Flp;+/+; FRT82B Akt1^{q}/FRT82B Ubi-GFPₙₗₛ (e,g). hsp70-Flp; ey-Gal4>DI/+;FRT82B Akt1^{q}/FRT82B Ubi-GFPnls (h) and ey-Flp; ey-Gal4>DI/+; FRT82B Akt1^{q}/FRT82B arm-lacZ (i). Homozygous mutant (Akt1⁻/Akt1⁻) clones were identified by the lack of Ubi-GFPₙₗₛ (GFP, green) or β-galactosidase (lacZ in green) marker, while associated control (+/+) sibling clones were distinguished from more strongly labeled GFP or β-galactosidase due to the presence of two copies of the Ubi-GFPnls or arm-lacZ marker construct, respectively. Cycling cells were marked by phospho-histone H3 (pH3) and eye field and photoreceptor differentiation were labeled using the Elav antibodies, respectively.

To measure area (FIG. 13j) of homozygous mutant Akt1⁻ and control (Akt1^{+/+}) sibling clones, mosaic discs each containing more than 15 clones were imaged on a Leica TCS-NT Confocal microscope and total areas of lacZ-negative (Akt1⁻/ Akt1⁻) and 2xlacZpositive (+/+ clones) tissue in each mosaic discs were determined using the ImageJ programme. n=14 mosaic discs of wt background scored, and n=21 mosaic discs of ey-Gal4>DI background scored. Clonal area measurements represent mean values (pixels ×10⁴ where 1 pixel=0.35 microns) of each genotype.

Western Blot of Drosophila eye discs. Eye disc lysates were obtained in 50 mM Tris, pH 8, 150 mM NaCl, 1% NP-40, 2 mM DTT, 10 mM NaF, 2 mM Pefablock, 2 mM NaVO4 and a cocktail of protease inhibitors (Roche). 20 Pg of each discs lysate was subjected to SDS-PAGE under reducing conditions and transferred onto an Immobilon polyvinylidene difluoride membrane (Millipore, Bedford, Mass.). After blocking with 3% of non-fat dry milk in PBS 0,1% Tween 20, protein detection was performed using the SuperSignal West Pico Chemiluminiscent system (Pierce). Detection of phospho Drosophila Akt (Ser505) was carried out using specific polyclonal antibodies rabbit antipAkt (Cell Signalling). Ser505 is a major site of Drosophila Akt phosphorylation and is homologous to mammalian Ser473.

Immunohistochemistry analysis of Drosophila eyes. The primary antibodies used were: mAb Elav at a dilution of 1:100, which labels differentiating photoreceptor cells; mAb Eya 1:100, which labels the eye primordium; anti-phosphorylated H3-S10 1:5000 (Upstate), which labels cells in mitosis; and rabbit anti-E-galactosidase (Cappel) 1:500. The secondary antibodies used were Alexa Fluor-596- and -488-conjugated (Molecular Probes) and Cy5-conjugated (Jackson lmmunoresearch) at 1:200. Images were captured on a Leica TCS-NT Confocal microscope.

Results of GSI Inactivation of NOTCH1 Signaling in GSI-Sensitive and -Resistant Cell Lines.

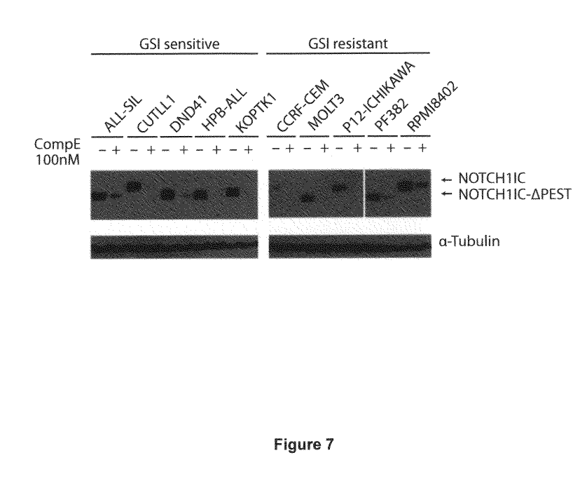

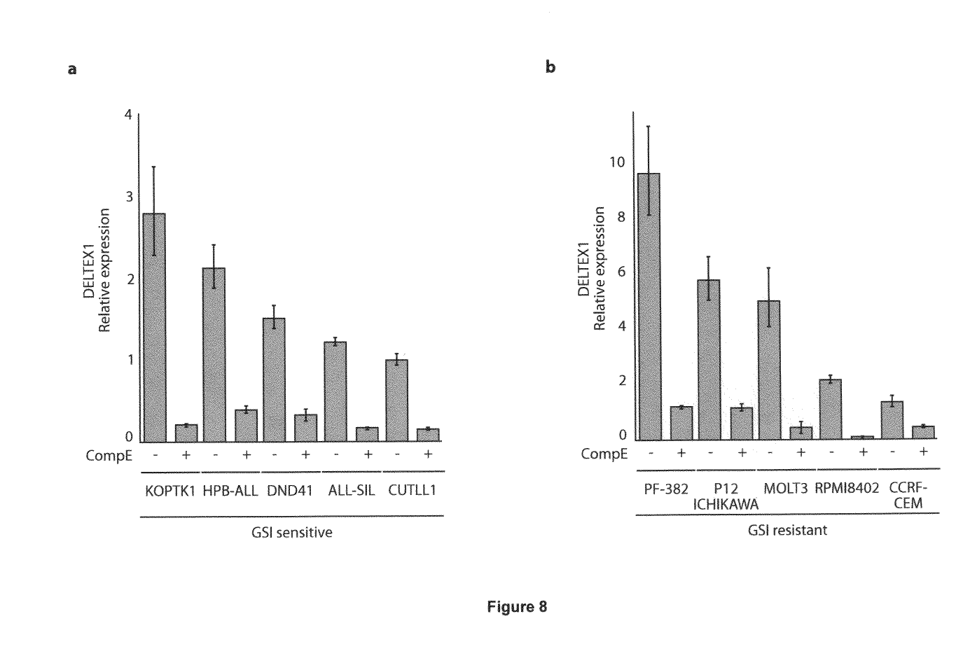

Western blot analysis with the Val1744 NOTCH1 antibody, which specifically recognizes the γ-secretase-cleaved activated form of NOTCH1 (NOTCH1IC) after treatment with CompE, a highly effective GSI, showed that CompE can effectively block NOTCH1 processing in all cell lines and that NOTCH1IC is readily cleared in both GSI-sensitive and GSI-resistant cells (FIG. 7). Furthermore, quantitative RT-PCR analysis of DELTEX1, a well-known NOTCH1 direct target gene, showed that CompE treatment effectively blocks NOTCH1 signaling and downregulates the expression of genes directly regulated by NOTCH1 (FIG. 8). These results support that decreased intracellular drug concentrations due to decreased uptake, increased export or increased drug metabolism; mutations in the γ-secretase complex inducing decreased affinity for the drug; or alternative (γ-secretase-independent) mechanisms of NOTCH1 activation, such as the expression of truncated intracellular forms of the NOTCH1 receptor, are not responsible for GSI resistance in most T-ALL cells.

GSI Treatment Phenocopies NOTCH Eye and Wing Defects and Reverts the Effects of Delta Overexpression in Drosophila.

Loss of function alleles of NOTCH result in characteristic wing and eye defects (e.g., tiny wing size and loss of wing margin and small eye defect) while generalized overexpression of the NOTCH ligand, Delta, in the developing eye by the ey-Gal4 produces mild eye overgrowth. Treatment with the GSI DAPT resulted in wing defects equivalent to a genetic reduction of NOTCH activity and resulted in suppression of eye overgrowth caused by high levels of expression of Delta (FIG. 5 and FIG. 16).

Analysis of Growth Phenotype Associated with Akt1 Loss- and Gain-of-Function in a Delta Gain-of-Function Background.

The adult fly eye develops from the eye imaginal disc, which proliferates extensively during the three larval stages. The growth of the eye depends on NOTCH activation in the dorsal-ventral organizer by its ligands Delta (human counterparts, DLL1, -3, -4) and Serrate (human counterparts, JAGGED-1, -2). The Drosophila phosphoinositide-3-OH-kinase-dependent serine/threonine protein kinase Akt1 affects cell and eye size in a cell autonomous manner, but its relationship with NOTCH-induced growth is unknown.

To determine the effect of complete loss of PI3K/Akt pathway activity on the Delta-induced growth in vivo, two techniques were employed to delete Akt1 function in a Delta overexpression (ey-Gal4>DI/+) background and in control wild type (+/+) background. The first technique employed heat-shock induction of the FLP recombinase to generate random mitotic clones of Akt1⁻ cells (Akt1^{q}) at a precisely defined time point. The comparison of clonal area of Akt1⁻ clones (marked by lack of GFP) and the clonal area of an associated “sibling clone” generated in the same recombination event and marked by two copies of GFP allowed an accurate assessment of the growth effect of Akt1 loss.

In contrast to previous reports that suggest that Drosophila Akt1 affects cell size without altering cell number, it was found that Akt1 affects cell number (FIGS. 13(e)-(g) and (j)). Clones homozygous for Akt1 contain fewer cells than the associated sibling clones and the mitosis marker (phospho H3) were found rarely within the mutant cells (FIG. 13(f)-(g)). Together with data shown in FIG. 5(h), this result demonstrates that Drosophila Akt1, as its mammalian counterpart, affects organ size by increasing cell growth and cell proliferation. Importantly, clones of Akt1⁻ cells in a Delta overexpression background were still markedly smaller than the sibling (Akt1^{+/+}) clones (FIG. 13(h)).

The second approach utilized the eye-specific ey-Flp recombinase to induce the mitotic clones. Since ey-Flp expresses continuously from the initial stages of eye disc formation until cell proliferation ceases in the disc, this enabled the recapitulation of the effects of deleting Akt1 throughout the entire proliferating phase of the eye disc (approximately ten cell cycles). Using this technique, clones of cells mutant for genes that do not affect cell proliferation normally occupy 50-70% of the discs. In contrast, clones of Akt1 were still very small (FIG. 13(i)). Quantification of total eye disc clonal areas is shown in FIG. 13(j). Clonal area is expressed in pixels×10⁴ (1 pixel=0.35 microns). In Delta overexpresion discs, the total area of Akt1⁻ clones were slightly larger in size (P<0.0001, n=21 mosaic discs scored) than Akt1⁻ clones in the wild type condition (n=14 mosaic discs scored), but still they were markedly smaller than the control (Akt1^{+/+}) sibling areas. Note that clonal areas of control (Akt1^{+/+}) sibling clones are slightly larger in the Delta overexpression background than in the wild type condition (P=0.0487) consistent with the role of Delta-NOTCH pathway in promoting cell proliferation in the eye disc.

The expression pattern of Akt1 phosphorylation was next examined by using a commercial rabbit polyclonal antibody that specifically detects Drosophila Akt1 when phosphorylated at serine 505. Whole-mount immunostainings of eye discs (FIG. 15(a)-(d)) showed that phospho Akt1 activation was increased when Delta was overexpressed in vivo. To assess the specificity of the pAkt antibody in immunostainings, mosaic eye discs containing Akt1⁻ clones were stained. Note that all available loss-of-function Akt1⁻ alleles are not protein-null. In particular, the allele Akt1^{q} encodes an inactive form of the Akt1 kinase carrying an amino acid replacement F327I in a core residue in the subdomain VII of the kinase catalytic domain, forming the DFG motif. As shown in FIG. 15 (e)-(f), anti-phospho Akt1 expression increases cell-autonomously within the clones of Akt1^{q} mutant cells marked by the lack of GFP. These findings are consistent with the dysregulation of negative feed-back mechanisms controlling Akt phosphorylation upon loss of Akt activity in the cell. A similar effect (upregulation of AKT phosphorylation at serine 473) has been observed in cells expressing a mammalian AKT mutant that impairs AKT activation by PDK. These results together with the detection of specific bands in Western blot analysis (data not shown) confirm the specificity of the p-Akt Ser 505 antibody. Collectively, these data along with the results shown in FIG. 5, show that NOTCH activation is associated with upregulation of the PTEN/PI3K/AKT signaling pathway and promotes cell growth in the Drosophila eye.

Targeted therapies for human tumors rely on the “addiction” of cancer cells to oncogenic signals driving tumor cell growth, proliferation, and survival. Thus, the oncogenic stimulus provided by activation of cellular oncogenes or the loss of tumor suppressors alters the circuitry maintaining normal cell-homeostasis in a way that makes the reprogrammed cells irreversibly dependent on high levels of oncogenic signaling for survival. The NOTCH1 signaling pathway is a critical controller of cell fate decisions and a key regulator of cell growth and metabolism during T-cell development and transformation. Several lines of evidence indicate that activation of PI3K-AKT signaling is a major downstream outcome of NOTCH1 signaling. Early work by Sade and coworkers showed that the Src family protein tyrosine kinase p56lck is required for NOTCH1-mediated activation of AKT in T-cells. However, we have failed to detect significant LCK activity in human T-ALL cell lines with constitutively active NOTCH1 signaling and high levels of AKT phosphorylation (data not shown), suggesting that LCK-mediated activation of AKT downstream of NOTCH1 does not occur in most human T-ALLs.

More recently, Ciofani and coworkers have shown that NOTCH signals regulate the trophic state (cell size, glucose uptake and glycolysis) of T-cell precursors through activation of the PI3K-Akt signaling pathway. We have recently identified a feed forward loop transcriptional network mediated by MYC promoting leukemic cell growth downstream of NOTCH140. Thus, multiple targets downstream of NOTCH1 organized in complex regulatory networks probably contribute to the pleiotrophic effects of NOTCH1 activation.

In the examples above, it was shown that NOTCH1 controls a transcriptional network regulating PTEN expression and the activity of the PI3K-AKT signaling pathway in normal thymocytes and in leukemic T cells. NOTCH signaling and the PI3K-AKT pathway synergize in vivo in a Drosophila model of NOTCH-induced tumorigenesis. Importantly, activation of AKT reverts a cell growth defect phenotype induced by the loss of NOTCH signaling in this model. Similarly, mutational loss of PTEN and consequent hyperactivation of AKT is associated with resistance to NOTCH1 inhibition in human TALL.

Overall, the above examples suggest that an evolutionally conserved regulatory circuit linking NOTCH1 signaling and AKT activity plays a key role in NOTCH1-induced transformation and mediates, at least in part, the cellular response to NOTCH1 inhibitors in T-ALL. According to this model, mutational loss of PTEN in T-ALL would turn this moderate and tightly controlled upregulation of PI3K-AKT downstream of NOTCH1, into an aberant and constitutively active oncogenic signal that would support cell growth and metabolism independently of NOTCH1 in PTEN-null T-ALL cells treated with GSIs (FIG. 6(a)). Finally, the foregoing examples show that bypass of dependence on aberrant NOTCH1 signaling for leukemic cell-growth by mutational loss of PTEN induces an oncogene addiction switch that renders T-ALL cells addicted to constitutively active AKT signaling.

Although illustrative embodiments of the present invention have been described herein, it should be understood that the invention is not limited to those described, and that various other changes or modifications may be made by one skilled in the art without departing from the scope or spirit of the invention.

1. A method of determining whether reducing or blocking NOTCH-1 activation will be effective to treat, prevent, or ameliorate the effects of a cancer in a patient comprising determining if the patient harbors one or more mutations in a PTEN coding region.

2. The method of claim 1, wherein NOTCH-1 activation is reduced or blocked by providing the patient with one or more γ-secretase inhibitors.

3. The method according to claim 2, wherein the γ-secretase inhibitors are selected from the group consisting of [(2S)-2-{[(3,5-Difluorophenyl)acetyl]amino}-N-[(3S)1-methyl-2-oxo-5-phenyl-2,3-dihydro-1H-1,4-benzodiazepin-3-yl]propanamide], N-[N-(3,5-difluorophenacetyl)-L-alanyl]-Sphenylglycine-t-butylester, and salts, and combinations thereof.

4. The method of claim 1, wherein the cancer is selected from the group consisting of T-cell leukemia, myeloleukemia, neuroblastoma, breast cancer, and ovarian cancer.

5. The method according to claim 4, wherein the cancer is T-cell leukemia.

6. The method of claim 1, wherein one or more mutations in a PTEN coding region is detected by (a) extracting DNA from the patient, (b) amplifying a portion of said DNA that comprises the PTEN coding region to produce an amplicon, and (c) sequencing the amplicon and determining whether the amplicon comprises one or more mutations in the PTEN coding region.

7. The method of claim 6, wherein the amplicon is produced using a pair of PCR primers consisting of SEQ ID NO:1 and SEQ ID NO:2.

8. The method of claim 1, wherein one or more mutations in a PTEN coding region is detected by (a) extracting DNA from the patient and (b) determining whether portions of the DNA in which the PTEN coding region resides hybridizes to one or more polynucleotides that are complementary to mutated forms of the PTEN coding region.

9. The method of claim 8, wherein a southern blot or microarray analysis is carried out to determine whether the patient harbors one or more mutations in a PTEN coding region.

10. The method of claim 1, wherein one or more mutations in a PTEN coding region is detected by (a) extracting DNA from the patient and (b) determining whether portions of the DNA in which the PTEN coding region resides hybridizes to one or more polynucleotides that are complementary to normal forms of PTEN.

11. The method of claim 10, wherein a southern blot or microarray analysis is carried out to determine whether the patient harbors one or more mutations in a PTEN coding region.

12. The method of claim 1, wherein one or more mutations in a PTEN coding region is detected by (a) reverse transcribing RNA that has been isolated from the patient into cDNA and (b) sequencing the cDNA and determining whether the amplicon comprises one or more mutations in the PTEN coding region.

13. The method of claim 1, wherein one or more mutations in a PTEN coding region is detected by measuring PTEN protein levels in a body fluid that is obtained from the patient.

14. The method of claim 13, wherein PTEN protein levels are measured using a procedure selected from the group consisting of immunoblots, ELISAs, RIAs, flow cytometry, and combinations thereof.

15. A method of determining whether an AKT inhibitor will be effective to treat, prevent, or ameliorate the effects of a cancer in a patient comprising determining if the patient harbors one or more mutations in a PTEN coding region.

16. The method of claim 15, wherein the AKT inhibitor is a phosphatidylinositol analog.

17. The method of claim 16, wherein the AKT inhibitor is SH-6.

18. The method of claim 15, wherein the cancer is T-cell leukemia.

19. A method of treating, preventing, or ameliorating the effects of a cancer in a patient comprising determining if the patient harbors one or more mutations in a PTEN coding region and (a) providing the patient with an AKT inhibitor if the patient harbors said mutations or (b) reducing or blocking NOTCH-1 activation in the patient if the patient does not harbor said mutations.

20. The method of claim 19, wherein NOTCH-1 activation is reduced or blocked by providing the patient with one or more γ-secretase inhibitors.

21. The method according to claim 20, wherein the γ-secretase inhibitors are selected from the group consisting of [(2S)-2-{[(3,5-Difluorophenyl)acetyl]amino}-N-[(3S)1-methyl-2-oxo-5-phenyl-2,3-dihydro-1H-1,4-benzodiazepin-3-yl]propanamide], N-[N-(3,5-difluorophenacetyl)-L-alanyl]-Sphenylglycine-t-butylester, and salts, and combinations thereof.

22. The method of claim 19, wherein the AKT inhibitor is a phosphatidylinositol analog.

23. The method of claim 22, wherein the AKT inhibitor is SH-6.

24. The method of claim 19, wherein the cancer is selected from the group consisting of T-cell leukemia, myeloleukemia, neuroblastoma, breast cancer, and ovarian cancer.

25. The method according to claim 24, wherein the cancer is T-cell leukemia.

26. A method for identifying whether a patient is resistant to a γ-secretase inhibitor comprising determining whether the patient has a mutation in a PTEN gene.

27. The method according to claim 26, wherein the determining step comprises carrying out a high throughput screening assay to determine whether a PTEN mutation is present in a sample of the patient's DNA.

28. A method for identifying whether a patient is sensitive to an AKT inhibitor comprising carrying out a screen for PTEN mutations on a sample of DNA from the patient, wherein the presence of a PTEN mutation in the DNA sample is indicative of the patient being sensitive to an AKT inhibitor.

29. A method for identifying a patient population for inclusion in a clinical trial of a drug candidate for treating cancer comprising carrying out a screen for PTEN mutations on a sample of DNA from each prospective patient, wherein the presence of a PTEN mutation in a patient's DNA sample is indicative of that patient being resistant to γ-secretase inhibitors and sensitive to AKT inhibitors, determining whether to include each patient in the clinical trial based on the patient's PTEN mutation status determined by the screen and the method of action of the drug candidate.
